(19) United States
(12) Patent Application Publication
(10) Pub. No.: US 2011/0218512 A1
(43) Pub. Date: Sep. 8, 2011

(54) ENHANCED ANTIVIRAL THERAPY METHODS AND DEVICES

(75) Inventors: Richard H Tullis, Encinitas, CA (US); Harold H. Handley, JR., Encinitas, CA (US); Paul R. Duffin, San Diego, CA (US); James A. Joyce, San Diego, CA (US)

(73) Assignee: Aethlon Medical, Inc., San Diego, CA (US)

(21) Appl. No.: 12/996,000

(22) PCT Filed: Jun. 3, 2009
(86) PCT No.: PCT/US09/46123

§ 371 Date: May 26, 2011

Related U.S. Application Data
(60) Provisional application No. 61/058,536, filed on Jun. 3, 2008.

(51) Int. Cl. A61M 1/36

(52) U.S. Cl. 604/500

(57) ABSTRACT

Embodiments of the present invention relate to enhanced antiviral therapy methods, devices, and kits for treating viral infections. The disclosed enhanced antiviral therapy methods, devices, and kits enhance the efficacy of an antiviral therapy by administering a lectin affinity hemodialysis treatment to an individual suffering from viral infection in combination with the antiviral therapy.

### RELATED APPLICATIONS

The instant application claims priority to U.S. Provisional Application No. 61/058,536 filed on Jun. 3, 2008, which is herein incorporated by reference in its entirety.

### BACKGROUND

1. Field of the Invention

Embodiments of the present invention relate to enhanced antiviral therapy methods, devices and kits for treating viral infections.

2. Description of the Related Art

A large number of viruses have been described which are pathogenic for humans. Viruses such as ebola, marburg, smallpox, lassa, dengue, influenza (e.g. H5N1 and H1N1), measles, mumps, viral encephalitis (e.g. Japanese encephalitis), HIV, hepatitis, herpes, and human cytomegalovirus (HCMV) are the etiological agents for debilitating and often incurable medical ailments. Aside from natural infection, the emerging threat of bioterror makes mass infections with these deadly agents ever more likely. Therapy is difficult for viral diseases as antibiotics have no effect on viruses and few antiviral drugs are known. In cases where drug treatments are available, the occurrence of resistant mutations and drug side effects often limit the effectiveness of therapy. Examples of such viruses include Hepatitis C virus (HCV) and human immunodeficiency virus (HIV). The best way to prevent viral diseases is through vaccination; however, vaccines are unavailable for a large number of viruses, including many of the viruses listed above. Although there are vaccines present for others, many available vaccine strategies are either not fully effective, as in the case of Hepatitis B Virus (HBV), or present potentially life-threatening side-effects, such as the vaccine released and recalled for rotavirus. Further, where vaccines do exist they are predominantly preventive and largely ineffective once a viral infection becomes established in the host.

Extracorporeal treatments provide a therapeutic modality which can be used to treat systemic disease. Extracorporeal perfusion of plasma over protein A, plasmapheresis and lymphapheresis have all been used as immunomodulatory treatments for HIV infection, and the thrombocytopenia resulting from it (Kiprov et al. Curr Stud Hematol Blood Trans/us 57: 184-197, 1990; Mittelman et al. Semin Hematol 26(2 Suppl 1): 15-18, 1989; Snyder et al. Semin Hematol 26(2 Suppl 1): 31-41, 1989; Snyder et al. Aids 5(10): 1257-1260, 1991). These therapies are all proposed to work by removing immune complexes and other humoral mediators, which are generated during HIV infection. They do not directly remove HIV virus. Extracorporeal photopheresis has been tested in preliminary trials as a mechanism to limit viral replication (Bisaccia et al., J Acquir Immune Defic Syndr 6(4): 386-392, 1993; Bisaccia et al., Ann Intern Med 113(4): 270-275, 1990). However, none of these treatments effectively remove both virus and viral proteins.

Chromatographic techniques for the removal of HIV from blood products have been proposed. In 1997, Motomura et al. proposed salts of a sulfonated porous ion exchanger for removing HIV and related substances from body fluids (U.S. Pat. No. 5,667,684). Takashima and coworkers (U.S. Pat. No. 5,041,079) provide ion exchange agents comprising a solid substance with a weakly acidic or weakly alkaline surface for extracorporeal removal of HIV from the body fluids of a patient. Both are similar to the work of Porath and Janson (U.S. Pat. No. 3,925,152) which described a method of separating a mixture of charged colloidal particles, e.g., virus variants by passing the mixture over an adsorbent constituted of an insoluble, organic polymer containing amphoteric substituents composed of both basic nitrogen-containing groups and acidic carboxylate or sulphonate groups (U.S. Pat. No. 3,925,152). However, none of these chromatographic materials are selective for viruses and will clearly remove many other essential substances. Thus they are not useful for in vivo blood purification.

Immunosorptive techniques have also been proposed for the treatment of viral infections. In 1980, Terman et al. described a plasmapheresis apparatus for the extracorporeal treatment of disease including a device having an immunoadsorbent fixed on a large surface area spiral membrane to remove disease agents (U.S. Pat. No. 4,215,688). The device envisioned no method for directly treating blood and required the presence of an immunologically reactive toxic agent. In 1991, Lopukhin et al. reported that rabbit antisera raised against HIV proteins, when coupled to Sepharose 4B or silica, could be used for extracorporeal removal of HIV proteins from the blood of rabbits which had been injected with recombinant HIV proteins (Lopukhin et al. Vestn Akad Med Nauk SSSR 11: 60-63, 1991). However, this strategy was inefficient as it required extracorporeal absorption of blood and did not provide for a mechanism to remove free HIV viral particles from the blood (Lopukhin et al., 1991, supra). U.S. Pat. No. 6,528,057 describes the removal of virus and viral nucleic acids using antibodies and antisense DNA. Plasmapheresis methods using lectins to remove virus and toxic viral proteins have also been described (U.S. Pat. No. 7,226,429). Other Plasmapheresis techniques have been described that employ antibodies to remove biological pathogens (U.S. Pat. No. 4,787,974).

Lectins are proteins that bind selectively to polysaccharides and glycoproteins and are widely distributed in plants and animals. Although many are insufficiently specific to be useful, it has recently been found that certain lectins are highly selective for enveloped viruses (De Clercq. et al Med Res Rev 20(5): 323-349, 2000). Among lectins which have this property are those derived from Galanthus nivalis in the form of Galanthus nivalis agglutinin (“GNA”), Narcissus pseudonarcissus in the form of Narcissus pseudonarcissus agglutinin (“NPA”) and a lectin derived from blue green algae Nostoc ellipsosporum called “cyanovirin” (Boyd et al. Antimicrob Agents Chemother 41(7): 1521-1530, 1997; Hammar et al. Ann N Y Acad Sci 724: 166-169, 1994; Kaku et al. Arch Biochem Biophys 279(2): 298-304, 1990). GNA is non-toxic and sufficiently safe that it has been incorporated into genetically engineered rice and potatoes (Bell et al. Transgenic Res 10(1): 35-42, 2001; Rao et al. Plant J 15(4): 469-477, 1998). These lectins bind to glycoproteins having a high mannose content such as found in HIV surface proteins (Chervenak et al. Biochemistry 34(16): 5685-5695, 1995). GNA has been employed in ELISA to assay HIV gp120 in human plasma (Hinkula et al. J Immunol Methods 175(1): 37-46, 1994; Mahmood et al. J Immunol Methods 151(1-2): 9-13, 1992; Sibille et al. Vet MicrobioI 45(2-3): 259-267, 1995) and feline immunodeficiency virus (FIV) envelope protein in serum (Sibille et al. Vet Microbiol 45(2-3): 259-267, 1995). Applicants have disclosed a method and device for using lectins that bind to virus having surface glycoproteins or fragments thereof which contain glycoproteins, to remove them from infected blood or plasma or other fluid. See U.S. Pat. No. 7,226,429.

The level of viral load is known to have an effect on virologic outcome. Arnaout et al. observed a negative correlation between viral load and survival time in HIV-1 infected patients. Arnaout et al. Proc. Natl. Acad. Sci. 1999, 96:11549-53. A study focusing on HIV antiretroviral therapy (ART) found that successfully treated patients had lower viral load values at study entry and that high baseline viral load were associated with clinical failure. Saag et al. Int Conf AIDS. 1998; 12: 336 (abstract no. 22363). At least one group has used a different virus-removal mechanism to reduce viral load in conjunction with an antiviral therapy. The group disclosed a double filtration plasmapheresis (DFPP) and interferon (IFN) (or a combination of IFN and ribavirin (RIB)) combination therapy for treating HCV patients with high viral load. Fujuwara et al., Hepatology Research 2007, 37:701-710.

However, there remains a need for the development of novel approaches to enhance the efficacy of known antiviral therapies for a broad spectrum of viral infections.

### SUMMARY OF THE INVENTION

Embodiments of the present invention relate to enhanced antiviral therapy methods, devices, and kits for treating viral infections. Embodiments of the disclosed enhanced antiviral therapy methods, devices, and kits enhance the efficacy of an antiviral therapy by administering a lectin affinity hemodialysis treatment to an individual suffering from viral infection prior to or in combination with the antiviral therapy.

One embodiment provides an enhanced antiviral therapy method for treating an individual suffering from viral infection, the method comprising: administering to said individual a course of antiviral therapy; and enhancing the efficacy of said antiviral therapy by administering a lectin affinity hemodialysis treatment to said individual, wherein said lectin affinity hemodialysis treatment comprises passing blood or plasma from said individual through a lectin affinity hemodialysis device, wherein a lectin in said lectin affinity hemodialysis device binds a virus or fragments thereof in said blood or plasma, and wherein said lectin traps said virus or fragments thereof in said lectin affinity hemodialysis device, removing said virus or fragments thereof from said blood or plasma. In some embodiment, the lectin affinity hemodialysis treatment is administered prior to administering said course of antiviral therapy. In some embodiments, the lectin affinity hemodialysis treatment is administered concurrent with the administering said course of antiviral therapy.

Another embodiment is an enhanced lectin affinity hemodialysis therapy method for treating an individual suffering from viral infection, the method comprising administering a course of lectin affinity hemodialysis therapy to the individual, where the lectin affinity hemodialysis therapy comprises passing blood or plasma from the individual through a lectin affinity hemodialysis device, where a lectin in the lectin affinity hemodialysis device binds a virus or fragments thereof in the blood or plasma, and where the lectin traps the virus or fragments thereof in the lectin affinity hemodialysis device, removing the virus or fragments thereof from the blood or plasma; and enhancing the efficacy of the course of lectin affinity hemodialysis therapy by administering to the individual a course of antiviral therapy during the course of the hemodialysis treatment. In some embodiments, the enhancement of the course of lectin affinity hemodialysis therapy comprises reducing the average viral load during the course of the lectin affinity hemodialysis therapy.

In some embodiments, the enhancing the efficacy of the antiviral therapy comprises increasing a rate at which the viral load of the patient is reduced during the administration of the course of antiviral therapy as compared to a viral load reduction rate achieved by administering either of the lectin affinity hemodialysis treatment or the course of antiviral therapy alone. In some embodiments, the rate at which the viral load of the individual is reduced is not less than 50%, 40%, 30%, or 20% higher as compared to the viral load reduction rate achieved by administering of the lectin affinity hemodialysis treatment alone combined with the viral load reduction rate achieved by administering the course of antiviral therapy alone. In some embodiment, the enhancing the efficacy of the antiviral therapy comprises reducing the amount of time required to achieve a clinically relevant viral load in the patient during the administration of the course of antiviral therapy as compared to the amount of time required to achieve said clinically relevant viral load by administering either of the lectin affinity hemodialysis treatment or the course of antiviral therapy alone.

One embodiment provides a kit for treating an individual suffering from viral infection, the kit comprising: a lectin affinity hemodialysis device; and at least one antiviral agent. In some embodiment, the kit further comprises an instruction for administering said antiviral agent. In some embodiment, the kit further comprises an instruction for using said lectin affinity hemodialysis device.

### BRIEF DESCRIPTION OF THE DRAWINGS

FIG. 1 is a schematic illustration of a longitudinal cross section of an embodiment of an affinity cartridge.

FIG. 2 is a schematic illustration of a horizontal cross section at plane 2 in FIG. 1.

FIG. 3 is an illustration of a channel from FIG. 2.

FIG. 4 is a schematic illustration of a conventional blood treatment system using the affinity cartridge of FIG. 1.

FIG. 5 is a schematic illustration of a blood treatment apparatus according to an embodiment.

FIG. 6 is a schematic illustration of a blood treatment apparatus according to an alternative embodiment.

### DETAILED DESCRIPTION OF THE PREFERRED EMBODIMENTS

The success rate of an antiviral therapy is thought to be correlated by the level of viral load in patients suffering from viral infection. A study of virologic response to antiretroviral triple drug therapy in HIV patients suggested that high baseline viral load is one of the independent risk factors for initial clinical failure. Zimmerli et al. Int Conf AIDS. 1998; 12: 333 (abstract no. 22349). In addition, low baseline viral load was found to be one of the predictors of success in the HIV antiretroviral therapy. Saag et al. Int Conf AIDS. 1998; 12: 336 (abstract no. 22363). Saag et al. showed that patients with lower viral load value at study entry had higher likelihood of virologic success and better clinical outcome and higher baseline viral load were associated with clinical failure. This study suggests that patients with lower viral load may respond to an antiviral therapy more quickly and/or better than the patients with higher viral load. Accordingly, embodiments of the present disclosure enhance the efficacy of an antiviral therapy by combining the antiviral therapy with a method that can physically remove virus and/or viral particles to reduce viral load. Lectin affinity hemodialysis treatment disclosed herein can improve the effectiveness and extend the benefit of an antiviral therapy compared to administering of either the lectin affinity hemodialysis treatment or the course of antiviral therapy alone.

The combination of the two therapies can have a number of benefits. When the hemodialysis treatment is administered less than continuously, (e.g. 4 to 8 hours a day, 1 to 7 times a week) there can be a rebound in the viral load between hemodialysis treatments. By combining hemodialysis with antiviral therapy, the rebound in viral load between hemodialysis treatments is reduced, resulting in a lower average viral load for the subject during the period of hemodialysis treatment plus antiviral therapy as compared to hemodialysis treatment alone. This can be seen as lower viral loads prior to the initiation of each individual hemodialysis treatment during the course of hemodialysis therapy, or in the average viral load during the hemodialysis therapy.

The combination of the two therapies can also result in the absence, or lessening of viral load rebound following the cessation of hemodialysis therapy. Hemodialysis therapy alone, or antiviral therapy alone, can achieve significant reductions in viral load. However, once the hemodialysis therapy or antiviral therapy is stopped, the viral load can begin to increase. This rebound in viral load can also be seen in patients that continue viral therapy as the virus adapts and becomes resistant to the antiviral being used. The combined therapy can reduce the level of rebound, preferably keeping it below a clinically or therapeutically relevant level. Alternatively, or in addition, the combination therapy can lengthen the amount of time before any rebound in viral load is seen.

In some embodiments, the improvement in the outcomes and rates discussed herein is less than additive, in some it is additive, and in some it is greater than additive, e.g., synergistic. In some embodiments, the rate at which the viral load of the patient is reduced is not less than about 5%, 10%, 15%, 20%, 25%, 30%, 35%, 40%, 45%, 50%, 55%, 60%, 65%, 70%, 75%, 80%, 90%, 100% higher as compared to the viral load reduction rate achieved by administering the lectin affinity hemodialysis treatment alone, as compared to the viral load reduction rate achieved by administering the course of antiviral therapy alone, or compared to the calculated combined rate of each therapy administered alone.

When the present disclosure refers to administering an antiviral therapy alone, it means that no lectin affinity hemodialysis treatment is administered to the patient prior to, during, or after the entire course of the antiviral therapy. When the present disclosure refers to administering a lectin affinity hemodialysis treatment alone, it means that no antiviral therapy is administered to the patient prior to, during, or after the entire course of the lectin affinity hemodialysis treatment.

Lectin-affinity hemodialysis devices described herein can be used to physically reduce viral load in a patient by directly removal of viruses and/or viral particles from blood or plasma of the patient before, during or after a course of antiviral therapy to enhance the efficacy of the antiviral therapy. When a patient suffering from viral infection receives a lectin affinity hemodialysis treatment prior to administering to the patient the course of antiviral therapy, the hemodialysis pre-treatment can lower the baseline viral load value for the antiviral therapy, so that the reduction rate of viral load during antiviral therapy may be increased compared to the administration of either lectin affinity hemodialysis treatment or the antiviral therapy alone. When the patient receives a lectin affinity hemodialysis treatment concurrently with the course of the antiviral therapy, the lectin affinity hemodialysis treatment can maintain the viral load in a stable reduced level during the course of the antiviral therapy treatment compared to the administration of either hemodialysis treatment or the antiviral therapy alone. When a patient receives a lectin affinity hemodialysis treatment after the patient has completed the antiviral therapy treatment, the hemodialysis treatment can further improve the clinical outcome of the antiviral therapy. Furthermore, lectin affinity hemodialysis treatment can help patients who are not responding or decreasing response to a known antiviral therapy by reducing viral load to a more manageable level for the antiviral therapy to achieve effectiveness.

In the enhanced antiviral therapy methods and associated devices and kits disclosed herein, lectin affinity viral hemodialysis devices described herein are used in combination with a conventional antiviral therapy thereby providing an improved and preferably synergistic reduction in viral load and clinical outcome. In some embodiments, the lectin-affinity hemodialysis therapy starts and completes before the commencement of the course of antiviral therapy. In some embodiments, the lectin-affinity hemodialysis therapy can include multiple session of hemodialysis over the course of days, weeks, or months. In some embodiments, the lectin-affinity hemodialysis therapy starts before the commencement of the course of antiviral therapy, continues during the course of the antiviral therapy, and completes before the end of the antiviral therapy. In some embodiments, the lectin-affinity hemodialysis therapy starts before the commencement of the antiviral therapy, continues during the course of antiviral therapy, and completes after the end of the antiviral therapy. In some embodiments, the lectin-affinity hemodialysis therapy starts before the commencement of the course of antiviral therapy, continues during the course of the antiviral therapy, and completes at the same time as the antiviral therapy. In some embodiments, the lectin-affinity hemodialysis therapy starts during the course of the antiviral therapy, continues during the course of antiviral therapy, and completes before the end of the antiviral therapy. In some embodiments, the lectin-affinity hemodialysis therapy starts during the course of antiviral therapy, continues during the course of antiviral therapy, and completes after the end of the antiviral therapy. In some embodiments, the lectin-affinity hemodialysis therapy starts during the course of antiviral therapy, continues during the course of antiviral therapy, and completes at the same time as the antiviral therapy. In some embodiments, the lectin-affinity hemodialysis therapy starts after the completion of the antiviral therapy. In some embodiments, the antiviral therapy is used in combination with the lectin affinity hemodialysis treatment to treat a patient suffering from a viral infection. In some embodiments, the patient is suffering from a pandemic flu strain.

Embodiments of the present invention relate to enhanced antiviral therapy methods for treating viral infection which use a lectin affinity hemodialysis treatment in combination with an antiviral therapy thereby providing an improved and preferably synergistic reduction in viral load. The lectin affinity hemodialysis treatment and devices described herein can remove viruses and fragments thereof from infected blood or plasma of a patient suffering from viral infection. Accordingly, some embodiments of the present disclosure provide an enhanced antiviral therapy method for treating an individual suffering from viral infection, the method comprises: administering to the individual a course of antiviral therapy and enhancing the efficacy of the antiviral therapy by administering a lectin affinity hemodialysis treatment to the patient. In some embodiments, the lectin affinity hemodialysis treatment is administered prior to the administration of the course of the antiviral therapy. In some embodiments, the lectin affinity hemodialysis treatment is administered concurrent with the administration of the course of the antiviral therapy. In some embodiments, the lectin affinity hemodialysis treatment enhances the efficacy of the antiviral therapy by reducing the viral load of the individual. In some embodiments, the enhancing the efficacy of the antiviral therapy comprises increasing the rate at which the viral load of the patient is reduced during the administration of the course of antiviral therapy as compared to a rate of viral load reduction achieved by administering either of the lectin affinity hemodialysis treatment or the course of antiviral therapy alone. In some embodiments, where the rate at which the viral load of the individual is reduced is not less than 50%, 40%, 30%, 20%, or 10% higher as compared to the viral load reduction rate achieved by administering of said lectin affinity hemodialysis treatment alone combined with the viral load reduction rate achieved by administering said course of antiviral therapy alone. In some embodiments, the enhancing the efficacy of the antiviral therapy comprises reducing the amount of time required to achieve a clinically relevant viral load in the individual during the administration of the course of antiviral therapy as compared to the amount of time required to achieve the clinically relevant viral load by administering either of the lectin affinity hemodialysis treatment or the course of antiviral therapy alone.

In any of the embodiments, the use of a lectin affinity device and antiviral therapy improves the effectiveness of the method or treatment compared to either the lectin affinity device or antiviral therapy alone. In a preferred embodiment, the improvement is additive, more preferably greater than additive, e.g. synergistic.

In some embodiments, a kit useful for practicing the methods described herein is provided. Such a kit generally comprises a lectin affinity viral hemodialysis device as described herein and at least one antiviral agent. In some embodiments, the antiviral agent can be any of the antiviral agents disclosed herein. In some embodiments, the kit contains instructions for administering the antiviral agent and/or using the lectin affinity hemodialysis device. The kit or any component of the kit can be presented in a commercially packaged form. The kit can be packaged in combination with one or more containers, devices, or necessary reagents and written or electronic instructions for the performance of the methods described herein. In some embodiments, the kit contains no less than one lectin-affinity cartridge and a daily dose of the antiviral agent. In some embodiments, the kit contains no less than one lectin-affinity cartridge and the antiviral agent sufficient for 3 days of treatment. In some embodiments, the kit contains no less than one syringe.

In some embodiments, the antiviral agent is selected from the group consisting of immunostimulators, immunomodulators, nucleoside antiviral agents, nucleotide antiviral agents, protease inhibitors, inosine 5′-monophosphate dehydrogenase (IMPDH) inhibitors, viral entry inhibitors, viral maturation inhibitors, viral uncoating inhibitors, integrase inhibitors, viral enzyme inhibitors, anti-sense molecules, ribozyme antiviral agents, nanoviricides, interferons and antibodies. In some embodiments, the antiviral agent is selected from the group consisting of amantadine, rimantadine, pleconaril, acyclovir, zidovudine, lamivudine, fomivirsen, zanamivir (Relenza) and oseltamivir (Tamiflu).

The antiviral agent can be stored in single-use vials or packages, or multiple-use vials or packages. The antiviral agent can be administered to the patient through: (a) oral pathways, which includes administration in capsule, tablet, granule, spray, syrup, or other such forms; (b) administration through non-oral pathways such as rectal, vaginal, intraurethral, intraocular, intranasal, or intraauricular, which includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the antiviral agent into contact with living tissue.

The methods disclosed herein can be applied to a wide spectrum of viruses, for example, any virus that can bind lectin is applicable. Any antiviral therapies known in the art (described in more details below) can be administered in combination with the lectin affinity hemodialysis treatment described in more details below and in related patents and patent applications: U.S. Pat. No. 7,226,429, PCT patent application No. PCT/US2008/087836, and PCT patent application No. PCT/US2008/063946. All the abovementioned references are incorporated by reference by their entirety.

The term “viral load” as used herein refers to the amount of viral particles or toxic fragments thereof in a biological fluid, such as blood or plasma. “Viral load” encompasses all viral particles, infectious, replicative and non-infective, and fragments thereof. Therefore, viral load represents the total number of viral particles and/or fragments thereof circulating in the biological fluid. Viral load can therefore be a measure of any of a variety of indicators of the presence of a virus, such as viral copy number per unit of blood or plasma or units of viral proteins or fragments thereof per unit of blood or plasma. Viral load can be determined by techniques known by one of skill in the art, e.g., polymerase-chain reaction (PCR) test and plaque-forming unit test. For example, viral load values can be determined by measuring the quantity of viral nucleic acid at the beginning of a treatment as well as at each of the virus measurement time points before, during, or after the treatment. A reduction in viral load during the course of treatment can be determined by comparing the viral load values obtained at different virus measurement time points. The rate at which the viral load of a patient is reduced can be determined by plotting the reduction in viral load value against time.

The term “viral load reduction rate” is defined as a rate at which the viral load is reduced, and refers to the amount of time required for an enhanced antiviral therapy, or an antiviral therapy, or a lectin affinity hemodialysis therapy to clear, or remove, a specific amount of viruses or viral particles from blood of a patient. For example, a system or treatment capable of reducing a viral load of 10×10⁹ copies by half (that is, to 5×10⁹ copies) in 1 hour has a viral load reduction rate of 5×10⁹ copies/hour (or 50% per hour), and a T_{1/2} or T_{50%} value of 1 hour. A system capable of reducing a viral load of 10×10⁹ copies by 90% (that is, to 1×10⁹ copies) in 1 hour has a viral load reduction rate of 9×10⁹ copies/hour (or 90% per hour), and a T_{90%} value of 1 hour.

In some embodiments, the reduction in viral load is measured by comparing the viral load of the patient immediately before the start of a lectin affinity hemodialysis session and the viral load of the patient immediately after the completion of that lectin affinity hemodialysis session. In some embodiments, the reduction in viral load is measured for every hemodialysis session during the course of a lectin affinity hemodialysis treatment or during the course of an enhanced antiviral therapy. In some embodiments, the reduction in viral load is measured every hour, every 4 hours, every 8 hours, every 12 hours, everyday, or every other day during the course of an antiviral therapy or during the course of an enhanced antiviral therapy. In some embodiments, the reduction in viral load follows a log linear clearance according to the formula: C=Co e^{-kt/V}, where C=virus concentration, k=constant of viral load reduction (=In²/t_{1/2;}); t_{1/2}=time to reduce the viral load by 50%; and V=blood volume of the patient. In some embodiments, the formula assumes a constant blood flow rate.

In some embodiments, the viral load reduction rate is, is about, is less than, is less than about, is more than, is more than about, 1×10⁴ copies/hour, 5×10⁴ copies/hour, 1×10⁵ copies/hour, 5×10⁵ copies/hour, 1×10⁶ copies/hour, 5×10⁶ copies/hour, 1×10⁷ copies/hour, 5×10⁷ copies/hour, 1×10⁸ copies/hour, 5×10⁸ copies/hour, 1×10⁹ copies/hour, 5×10⁹ copies/hour, 1×10¹⁰ copies/hour, 5×10¹⁰ copies/hour, 1×10¹¹ copies/hour, 5×10¹¹ copies/hour, 1×10¹² copies/hour, or 5×10¹² copies/hour, 1×10⁴ copies/day, 5×10⁴ copies/day, 1×10⁵ copies/day, 5×10⁵ copies/day, 1×10⁶ copies/day, 5×10⁶ copies/day, 1×10⁷ copies/day, 5×10⁷ copies/day, 1×10⁸ copies/day, 5×10⁸ copies/day, 1×10⁹ copies/day, 5×10⁹ copies/day, 1×10¹⁰ copies/day, 5×10¹⁰ copies/day, 1×10¹¹ copies/day, 5×10¹¹ copies/day, 1×10¹² copies/day, or 5×10¹² copies/day or a range defined by any two of these values. In some embodiments, the viral load reduction rate is, is about, is less than, is less than about, is more than, is more than about, 0.1% per hour, 0.25% per hour, 0.5% per hour, 1% per hour, 2.5% per hour, 5% per hour, 10% per hour, 15% per hour, 20% per hour, 25% per hour, 30% per hour, 40% per hour, 50% per hour, 60% per hour, 70% per hour, 80% per hour, or 90% per hour, or 0.1% per day, 0.25% per day, 0.5% per day, 1% per day, 2.5% per day, 5% per day, 10% per day, 15% per day, 20% per day, 25% per day, 30% per day, 40% per day, 50% per day, 60% per day, 70% per day, 80% per day, or 90% per day, or a range defined by any of these two values. In some embodiments, continuous reduction in viral load is performed with slower reduction rates (for example, 5% per hour or less), for up to 24 hours per day over one, two, three or more days or weeks. In some embodiments, T_{1/2} or T_{50%} is, is about, is less than, is less than about, is more than, is more than about, 15, 30, or 45 minutes, or 1, 2, 3, 4, 5, 6, 7, 8, 9, 10, 11, 12, 13, 14, 15, 16, 17, 18, 19, 20, 21, 22, 23, or 24 hours, or a range defined by any two of these values. In some embodiments, T_{90%} is, is about, is less than, is less than about, is more than, is more than about, 1, 2, 3, 4, 5, 6, 7, 8, 9, 10, 11, 12, 13, 14, 15, 16, 17, or 18 hours, or a range defined by any two of these values.

The term “sustained viral response (SVR)” as used herein refers to having a negative HCV viral load test (i.e., no detectable virus) 6 months after stopping HCV treatment. SVR test determines whether treatment has been effective in terms of clearing HCV. SVR is one of the important results from an HCV treatment trial.

The term “contaminant” as used herein includes but is not limited to biological pathogens, such as viral particles and fragments thereof, exosomes, as well as toxins, chemicals, heavy metals, drugs and chemotherapeutic agents. “Contaminant” encompasses any undesirable substance which may be found in a bodily fluid.

The terms “affinity-binding material,” “affinity-binding medium,” “affinity-binding agent,” and “contaminant-binding substrate” as used herein refer to any mechaism by which a targeted contaminant may be selectively trapped or bound and thereby removed from a fluid. “Affinity-binding material” “affinity-binding medium,” “affinity-binding agent,” and “contaminant-binding substrate” include, for example, activated charcoal, antibodies, and lectins, as well as materials in which or on which such substances may be disposed. Some examples of lectins include, without limitation, Galanthus nivalis agglutinin (GNA), Narcissus pseudonarcissus agglutinin (NPA), cyanovirin (CVN), Conconavalin A, Griffithsin and mixtures thereof.

The term “plaque forming units” or “pfu” as used herein refers to the amount of infectious virus particles in a biological fluid, such as blood or plasma. One plaque forming unit is equivalent to one infectious virus particle. In some embodiments, viral plaque forming units are more critical to reduce than viral load. One skilled in the art would recognize that there are several ways to determine the number of plaque forming units in a particular sample. See e.g., Lee H, and Jeong, Y S (2004) Comparison of Total Culturable Virus Assay and Multiplex Integrated Cell Culture-PCR for Reliability of Waterborne Virus Detection. Appl Environ OMicrobial. 2004 June; 70(6): 3632-3636. In one particular assay, cells are grown on a flat surface until they form a monolayer of cells covering a bottle or dish. They are then infected with the target sample, or a particular dilution thereof. A plaque is produced when a virus particle infects a cell, replicates, and lyses, killing the cell. Surrounding cells are infected by the newly replicated virus and they too are killed. This process can repeat several times, such that sufficient numbers of neighboring cells are infected and lysed to form a cell-free hole within the monolayer of cells. The cells can be stained with a dye which stains only living cells. The dead cells in the plaque do not stain and appear as unstained areas on a colored background. Each plaque is the result of infection of one cell by one virus followed by replication and spreading of that virus. However, viruses that do not kill cells can not produce plaques and can contribute to the viral load without affecting the pfu count.

The term “high mannose glycoprotein” as used herein for the purpose of the specification and claims refers to glycoproteins having mannose-mannose linkages in the form of α-1→3 or α-1→6 mannose-mannose linkages. Some examples of lectins which bind glycoproteins including high mannose glycoproteins include, without limitation, Galanthus nivalis agglutinin (GNA), Narcissus pseudonarcissus agglutinin (NPA), cyanovirin (CVN), ConconavalinA, Griffithsin and mixtures thereof.

The terms “total fluid flow rate,” “whole blood flow rate,” and “blood flow rate” as used herein refer to the volumetric flow rate of fluid flowing into the main flow path of the device prior to any subsequent separation or treatment. The term “main flow path” refers to the flow path through the device on the same side of the membrane as the inlet.

The terms “assisted flow rate,” “secondary flow rate,” and “plasma flow rate” as used herein refer to the volumetric flow rate of the fluid passing through the membrane and flowing in a secondary flow path. The terms “secondary flow path” and “plasma flow path” refer to the flow path through the device on the opposite side of the membrane as the inlet.

The term “exposed,” as used herein in the context of blood being “exposed” to any type of lectin-containing substrate, refers to any virus-containing portion of blood contacting a lectin-containing substrate. In some embodiments, the blood is exposed to the lectin-containing substrate for a specific amount of time. Exposure of the blood to the lectin-containing substrate, as used herein, refers to the total amount of time the blood is exposed to the lectin-containing substrate and not the amount of time blood is processed through the device. In some embodiments, the fluid is exposed to the contaminant-binding substrate for a specific amount of time.

The term “clearance rate,” as used herein, refers to the amount of time required to clear, or remove, a specified amount of contaminant from a volume of blood. The term “viral clearance rate,” as used herein, refers to the amount of time required for the lectin affinity hemodialysis device to clear, or remove, a specific amount of viruses or viral particles from a volume of blood. For example, a system capable of reducing a viral load of 10×10⁹ copies by half (that is, to 5×10⁹ copies) in 1 hour has a viral clearance rate of 5×10⁹ copies/hour (or 50% per hour), and a T_{1.2} or T_{50%} value of 1 hour. A system capable of reducing a viral load of 10×10⁹ copies by 90% (that is, to 1×10⁹ copies) in 1 hour has a viral clearance rate of 9×10⁹ copies/hour (or 90% per hour), and a T_{90%} value of 1 hour.

In some embodiments, the viral clearance rate is, is about, is less than, is less than about, is more than, is more than about, 1×10⁴ copies/hour, 5×10⁴ copies/hour, 1×10⁵ copies/hour, 5×10⁵ copies/hour, 1×10⁶ copies/hour, 5×10⁶ copies/hour, 1×10⁷ copies/hour, 5×10⁷ copies/hour, 1×10⁸ copies/hour, 5×10⁸ copies/hour, 1×10⁹ copies/hour, 5×10⁹ copies/hour, 1×10¹⁰ copies/hour, 5×10¹⁰ copies/hour, 1×10¹¹ copies/hour, 5×10¹¹ copies/hour, 1×10¹² copies/hour, or 5×10¹² copies/hour, 1×10⁴ copies/day, 5×10⁴ copies/day, 1×10⁵ copies/day, 5×10⁵ copies/day, 1×10⁶ copies/day, 5×10⁶ copies/day, 1×10⁷ copies/day, 5×10⁷ copies/day, 1×10⁸ copies/day, 5×10⁸ copies/day, 1×10⁹ copies/day, 5×10⁹ copies/day, 1×10¹⁰ copies/day, 5×10¹⁰ copies/day, 1×10¹¹ copies/day, 5×10 copies/day, 1×10¹² copies/day, or 5×10¹² copies/day or a range defined by any two of these values. In some embodiments, the viral clearance rate is, is about, is less than, is less than about, is more than, is more than about, 0.1% per hour, 0.25% per hour, 0.5% per hour, 1% per hour, 2.5% per hour, 5% per hour, 10% per hour, 15% per hour, 20% per hour, 25% per hour, 30% per hour, 40% per hour, 50% per hour, 60% per hour, 70% per hour, 80% per hour, or 90% per hour, or 0.1% per day, 0.25% per day, 0.5% per day, 1% per day, 2.5% per day, 5% per day, 10% per day, 15% per day, 20% per day, 25% per day, 30% per day, 40% per day, 50% per day, 60% per day, 70% per day, 80% per day, or 90% per day, or a range defined by any of these two values. In some embodiments, continuous clearance is performed with slower clearance rates (for example, 5% per hour or less), for up to 24 hours per day over one, two, three or more days or weeks. In some embodiments, T_{1/2} or T_{50%} is, is about, is less than, is less than about, is more than, is more than about, 15, 30, or 45 minutes, or 1, 2, 3, 4, 5, 6, 7, 8, 9, 10, 11, 12, 13, 14, 15, 16, 17, 18, 19, 20, 21, 22, 23, or 24 hours, or a range defined by any two of these values. In some embodiments, T_{90%} is, is about, is less than, is less than about, is more than, is more than about, 1, 2, 3, 4, 5, 6, 7, 8, 9, 10, 11, 12, 13, 14, 15, 16, 17, or 18 hours, or a range defined by any two of these values.

In some embodiments, the viral load or pfu/ml in the blood or plasma is reduced to a clinically relevant viral load. The term “clinically relevant viral load” as used herein refers to a viral load or pfu/ml in the blood or plasma that halts or slows the progression of the infection, and slows or prevents the worsening of symptoms associated with the infection, and preferably improves and eliminates the infection or symptoms thereof. In some cases, reducing viral load or pfu/ml by or to a “clinically relevant viral load” can allow an infected individual's immune system to maintain or reduce the viral load or pfu/ml without further intervention. In some embodiments, “clinically relevant viral load” is an amount sufficient to render another treatment (e.g. a drugs, retroviral therapy, etc.) effective, or more effective. The “clinically relevant viral load” can vary with different viruses and individuals, but can be readily determined by a skilled artisan. For example, for HIV infection current antiviral treatments have a target level of is no greater than about 1000 virus copies/ml (the undetectable level is usually below 50 copies/ml), whereas Ebola infected monkeys are said to resolve disease on their own if the count can be reduced below 50,000 copies/ml (as measured by quantitative RT-PCR). In some embodiments, the clinically relevant viral load is less than about 60000, 50000, 40000, 30000, 20000, 10000, 9000, 8000, 7000, 6000, 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2, or 1 copies/ml.

In some embodiments, the enhancing the efficacy of said antiviral therapy comprises increasing the rate at which the viral load of a patient reduced measured during the administration of the course of antiviral therapy as compared to the viral reduction rate achieved by administering either of said lectin affinity hemodialysis treatment or said course of antiviral therapy alone. In some embodiments, the increase in a rate at which the viral load of a patient is reduced during the administration of the course of antiviral therapy as compared to the viral reduction rate achieved by administering of either lectin affinity hemodialysis treatment or the course of antiviral therapy alone is at least about 1%, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%. In some embodiments, the rate at which the viral load of a patient is reduced during the administration of the course of antiviral therapy is higher than the viral reduction rate achieved by administering of lectin affinity hemodialysis treatment alone combined with the viral load reduction rate achieved by administering the course of antiviral therapy alone by a percentage of at least about 1%, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%. In some embodiments, the rate at which the viral load of a patient is reduced during the administration of the course of antiviral therapy is at least about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% per hour, per 8 hours, per 12 hours, or per day.

In some embodiments, enhancing the efficacy of said antiviral therapy is achieved by reducing the amount of time required to achieve a clinically relevant viral load in the patient during the administration of the course of antiviral therapy as compared to administering of either said lectin affinity hemodialysis treatment or said course of antiviral therapy alone. In some embodiments, the clinically relevant viral load is less than about 100000, 90000, 80000, 70000, 60000, 50000, 40000, 30000, 20000, 10000, 9000, 8000, 7000, 6000, 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2, or 1 copies/ml. In some embodiments, the amount of time required to achieve the clinically relevant viral load compared to administration of either the lectin affinity hemodialysis or the course of antiviral therapy alone is reduced by at least about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1%. In some embodiments, the amount of time required to achieve the clinically relevant viral load is less than about 36, 35, 34, 32, 31, 30, 29, 28, 27, 26, 25, 24, 23, 22, 21, 20, 19, 18, 17, 16, 15, 14, 13, or 12 months, or 56, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, or 4 weeks, or 28, 27, 26, 25, 24, 23, 22, 21, 20, 19, 18, 17, 16, 15, 14, 13, 12, 11, 10, 9, 8, 7, 6, 5, 4, 3, 2 or 1 days.

In some embodiments, the lectin affinity hemodialysis treatment is administered for about 1, 2, 3, 4, 5, 6, 7, 8, 9, 10, 11, 12, 13, 14, 15, 16, 17, 18, 19, 20, 21, 22, 23 or 24 hours during a 24 hour period. In some embodiments, the lectin affinity hemodialysis treatment is administered at a frequency of about once per week, twice per week, three times per week, four times per week, five times per week, six times per week, seven times per week, once every four days, once every three days, once every two days, once per day, twice per day, three times per day, or four times a day. In some embodiments, the lectin affinity hemodialysis treatment is administered no more than seven times, six times, five times, four times, three times, twice, or once per week.

### Antiviral Therapies

The enhanced antiviral therapy methods disclosed herein can be applied to any antiviral therapy. Conventional antiviral therapies include those now available are designed to help deal with HIV, herpes viruses, the hepatitis B and C viruses, and influenza A and B viruses. Researchers are now working to extend the range of antivirals to other families of pathogens.

In the methods disclosed herein, a course of antiviral therapy comprises administering to a patient at least one antiviral agent, typically once or more times a day for several days, weeks, months or even years. Antiviral agents include, but not limited to immunostimulants, immunosuppressants, nucleoside antiviral agents, nucleotide antiviral agents, protease inhibitors, inosine 5′-monophosphate dehydrogenase (IMPDH) inhibitors, viral entry inhibitors, viral maturation inhibitors, viral uncoating inhibitors, integrase inhibitors, viral enzyme inhibitors, antisense antiviral molecules, ribozyme antiviral agents, nanoviricides, interferons, and antibodies.

An immunostimulant is any substance (e.g., drugs and nutrients) that enhances or potentiates the immune system by inducing activation or increasing activity of any of its components Immunostimulants include specific immunostimulants and non-specific immunostimulants Immunostimulants include, but are not limited to interferon, granulocyte macrophage colony-stimulating factor, echinacin, isoprinosine, adjuvants, biodegradable microspheres (e.g., polylactic galactide) and liposomes (into which the compound is incorporated), and thymus factors. Interferon includes, but not limited to, alpha interferons, beta interferons, gamma inteferons, pegylated alpha interferons, pegylated beta interferons, pegylated gamma interferons and mixtures of any two or more thereof.

An immunosuppressant is any substance that suppresses the immune system. Non-limiting examples of immunosuppressants are: cyclosporin, azatioprin, methotrexate, cyclophsphamide, FK 506, cortisol, betametasone, cortisone, desametasone, flunisolide, prednisolone, methylprednisolone, prednisone, triamcinolone, alclometasone, amcinonide desonide, desoxymetasone, prednisone, cyclosporine, mycophenolate mofetil, and tacrolimus.

Nucleoside and nucleotide antiviral agents include, but not limited to, abacavir, acyclovir (ACV), adefovir, zidovudine (ZDV), ribavirin, lamivudine, adefovir and entecavir, tenofovir, emtricitabine, telbuvidine, clevudine, valtorcitabine, cidofovir, and derivatives thereof.

Protease inhibitors are molecules that inhibit the function of proteases. They are used to treat or prevent infection by viruses, including HIV and HCV. For example, an HIV protease inhibitor prevents viral replication by inhibiting the activity of HIV-1 protease, an enzyme used by the viruses to cleave nascent proteins for final assembly of new virons. Antiretroviral protease inhibitors for treating viral infection include, but are not limited to, saquinavir, ritonavir, indinavir, nelfinavir, amprenavir, atazanavir, boceprevir, and HCV NS3 protease inhibitors.

Ribozyme antiviral agents are synthetic enzymes that are designed to cut viral RNA or DNA at selected sites that will disable viruses. RNase P ribozyme is an antiviral agent against human cytomegalovirus.

Viral enzyme inhibitors have the ability to bind to enzymes produced by viruses in a way that disrupts their function, preventing a step in the infectious process from occurring. Viral enzyme inhibitors include, but are not limited to reverse transcriptase inhibitor (e.g., efavirenz), protease inhibitors, neuraminidase inhibitors (e.g., Zanamivir (Relenza) and Oseltamivir (Tamiflu)), RNaseH inhibitor, inhibitors of enzymes involved in viral entry, inhibitors of enzymes involved in viral maturation, inhibitors of enzymes involved in viral uncoating, and integrase inhibitors. Non-limiting examples include HCV NS5B polymerase inhibitor.

Inhibitors of viral uncoating and/or penetration include, but are not limited to, amantadine, rimantadine, pleconaril, and derivatives thereof.

Antisense antiviral molecules include, but are not limited to, oligonucleotides designed to recognize and inactivate viral genes. Antisense antiviral oligonucleotides can prevent viruses from replicating in the human body, and thus treat viral infections. A phosphorothionate antisense drug fomivirsen has been used to treat opportunistic eye infections in AIDS patients caused by cytomegalovirus.

Nanoviricides are known as polymeric micelles, nano-sized polymer structures that convey medicine in the bloodstream in a time-release fashion. In addition to delivering vaccines they can target, neutralize, dismantle and destroy viruses as well.

### Lectin Affinity Hemodialysis Devices

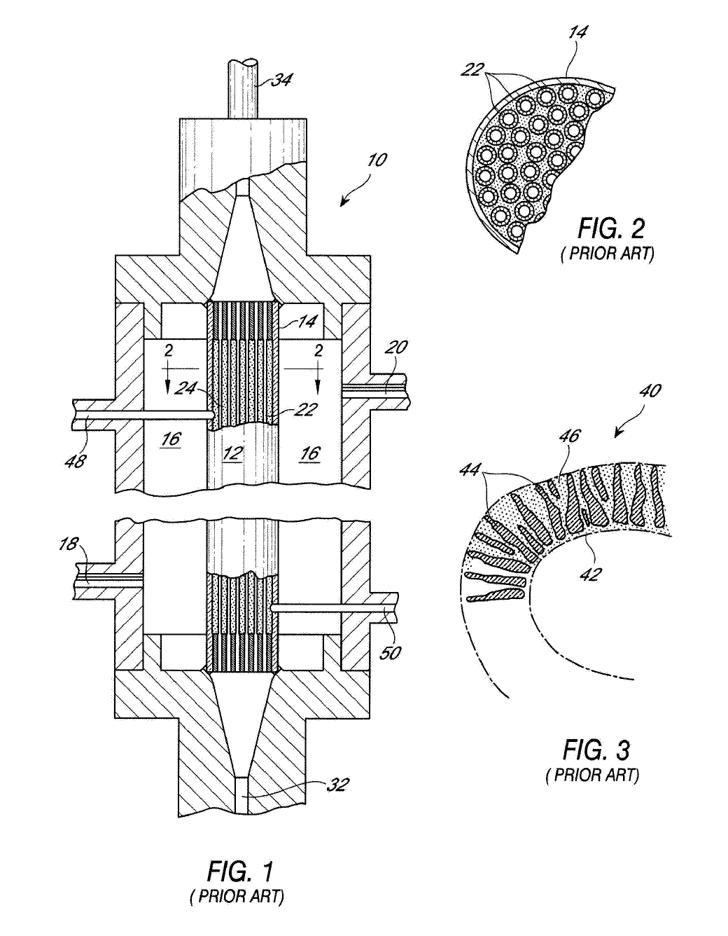

In some embodiments, the enhanced antiviral therapy method described herein is carried out by using an affinity cartridge as the device illustrated in FIG. 1 and described below in greater detail. Devices of this general type are disclosed in U.S. Pat. Nos. 4,714,556, 4,787,974 and 6,528,057, the disclosures of which are incorporated herein by reference in their entireties. In this device, blood is passed through the lumen of a hollow fiber membrane, wherein lectins are located in the extrachannel space of the cartridge, which form a means to accept and immobilize viruses and toxic andlor infectious fragments thereof. Thus, the device retains intact virions and viral glycoproteins bound by lectin while allowing other blood components to pass through the lumen. The device comprises a cartridge 10 comprising a blood-processing chamber 12 formed of interior glass or plastic wall 14. Around chamber 12 is an optional exterior chamber 16. A temperature controlling fluid can be circulated into chamber 16 through port 18 and out of port 20. The device includes an inlet port 32 for the blood and an outlet port 34 for the effluent. The device also provides one or more ports 48 and 50, for accessing the extrachannel or extralumenal space in the cartridge. FIG. 2 is a schematic illustration of a horizontal cross section at plane 2 in FIG. 1. As shown in FIGS. 1 and 2, chamber 12 contains a plurality of membranes 22. These membranes preferably have a 0.3 mm inside diameter and 0.5 mm outside diameter. In some embodiments, the outside or inside diameter is 0.025 mm to 1 mm more preferably 0.1 to 0.5 mm more preferably 0.2 to 0.3 mm, as close to the outside diameter as allowed to minimize flow path length while still providing structural integrity to the fiber. FIG. 3 is a cross sectional representation of a channel 22 and shows the anisotropic nature of the membrane. As shown in FIG. 3, a hollow fiber membrane structure 40 is preferably composed of a single polymeric material which is formed into a tubular section comprising a relatively tight plasmapheresis membrane 42 and relatively porous exterior portion 44 in which can be immobilized lectins 46. During the operation of the device, a solution containing the lectins is loaded on to the device through port 48. The lectins are allowed to immobilize to the exterior 22 of the membrane in FIG. 2. Unbound lectins can be collected from port 50 by washing with saline or other solutions. Alternatively, the lectins can be bound to a substrate which is loaded into the extrachannel or extralumenal space, either as a dry substance (e.g. sand), or in solution or slurry.

One embodiment of an affinity device, described in detail below with reference to FIGS. 1-3, includes multiple channels of hollow fiber membrane that forms a filtration chamber. An inlet port and an effluent port are in communication with the filtration chamber. The membrane is preferably an anisotropic membrane with the tight or retention side facing the bloodstream. The membrane is formed of any number of polymers known to the art, for example, polysulfone, polyethersulfone, polyamides, polyimides, and cellulose acetate. In other embodiments, the porous membrane is a sheet, rather than a channel. The sheet can be flat, or in some other configuration, such as accordion, concave, convex, conical, etc., depending on the device. In some embodiments, the membrane has pores with a mean diameter of, of about, of less than, of less than about, of more than, of more than about, 1950, 1900, 1850, 1800, 1750, 1700, 1650, 1600, 1550, 1500, 1450, 1400, 1350, 1300, 1250, 1200, 1150, 1100, 1050, 1000, 950, 900, 850, 800, 750, 700, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, or 85 nm, which will allow passage of intact viruses and viral particles and fragments (e.g., Rous Sarcoma Virus virions of 80 nm diameter, HCV of 50 nm), but not most blood cells. In other embodiments, the membrane has pores in a range between any two pore diameters recited above.

Preferably, the membrane has pores 200-500 nm in diameter, more preferably, the pore size is 600 nm, which will allow passage of intact viruses and viral particles and fragments (e.g., HIV virions of 110 nm diameter), but not most blood cells (red blood cells 10,000 nm diameter, lymphocytes 7,000-12,000 nm diameter, macrophages 10,000-18,000 nm diameter, thrombocytes 1000 nm). Optionally, by selecting a pore size that is smaller than the diameter of blood cells, the membrane excludes substantially all blood cells from passing through the pores and entering the extrachannel or extralumenal space of the device that contains the lectin. In some embodiments, a pore size is selected that is smaller than only some blood cell types.

In this device, the time of exposure is a function of the flow rate and the capacity of the lectin-binding substrate. For example, if the whole blood flow rate of a device is 10 ml/min and the capacity of the device is 10 ml, then running unprocessed blood for 30 minutes would expose 300 ml of blood to the lectin-containing substrate for 1 minute. For further illustration, if 30 ml of blood were recirculated over a device with the same flow rate and same capacity for 30 minutes, then the 30 ml of blood would be exposed to the lectin-containing substrate for 10 minutes. In some embodiments, the blood is exposed to a lectin-containing substrate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10 , 9, 8, 7, 6, 5, 4, 3, 2, or 1 minutes. In other embodiments, the time the blood is exposed to a lectin-containing substrate is a range defined by any two times recited above. In some embodiments, the capacity of the lectin-binding substrate can vary based on the extent of saturation of the lectin substrate.

In another embodiment, the blood flow rate into the device is about 60 ml/min to about 400 ml/min In another preferred embodiment, the flow rate through the device is about 250 ml/min to about 400 ml/min. In some embodiments, the flow rate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml/min., or a range defined by any two of these values. In some embodiments, the capacity of the device is 40 ml. Also contemplated are devices where the capacity is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml, or a range defined by any two of these values.

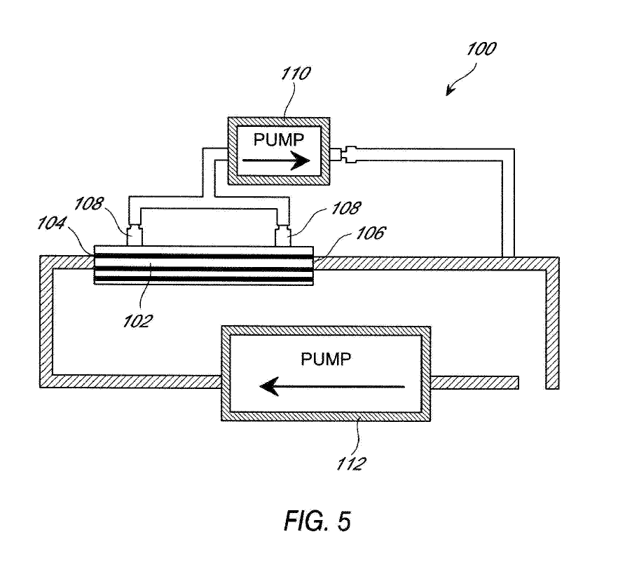

In another embodiment, an extracorporeal blood treatment apparatus 100 described in FIG. 5 is used. The apparatus 100 includes a plasma separator 102 having an inlet port 104, an outlet port 106, a main flow pump 112, and one, two or more plasma ports 108 in fluid communication with a plasma pump 110. The plasma separator 102 preferably comprises a separation membrane surrounded by a cartridge. The separation membrane has pores sized to allow passage of the plasma component of the blood across the membrane, while preventing passage of all of, nearly or substantially all of, a majority of, or a portion of, the cellular component of the blood, including blood cells and platelets. The separation membrane thus functions to separate a main flow path, running from the inlet port 104 on one side of the membrane to the outlet port 106 of the plasma separator 102, from a plasma flow path, beginning on the other side of the membrane and running through the plasma port(s) 108 to the plasma pump 110. To allow contaminants to pass across the separation membrane along with the plasma, the pores are preferably between about 100 nm and 200 nm in diameter, or other appropriate sizes, including those described elsewhere in the specification. The inlet port 104 and the outlet port 106 are in fluid communication with the main flow path, and the plasma ports 108 are in fluid communication with the plasma flow path. To withdraw fluid in an evenly distributed flow from the extralumenal space of the cartridge and prevent accumulation and clogging of the plasma ports with matrix/substrate material, the plasma ports 108 are preferably provided with wicks configured to draw fluid from inside the separator cartridge through the plasma ports 108. The main flow pump 112 is preferably located upstream of the separator 112, but can be located downstream of the separator.

In a preferred embodiment, the separation membrane comprises one or more hollow fiber membranes. In embodiments comprising hollow fiber membranes, the inlet port 104 and the outlet port 106 are in fluid communication with the lumens of the hollow fiber membranes, which define a portion of the main flow path through the apparatus. The plasma ports 108 are in fluid communication with the extralumenal space surrounding the hollow fibers within the separator cartridge. Thus, the hollow fiber membranes separate the main flow path from the start of the plasma flow path of the apparatus 100. The hollow fiber membranes preferably have a 0.3 mm inside diameter and 0.5 mm outside diameter. In some embodiments, the outside or inside diameter is 0.025 mm to 1 mm, more preferably 0.1 to 0.5 mm, or even more preferably 0.2 to 0.3 mm In some embodiments, the cartridge 102 includes lectins or other affinity binding materials immobilized in the extralumenal space, as described above in connection with FIGS. 1-3.

With continued reference to FIG. 5, the plasma pump 110 can comprise, for example, a negative pressure pump configured to assist the flow of plasma crossing the separation membrane and traveling through the extralumenal space containing the pathogen binding lectins, thereby increasing contact between the plasma and the lectins and increasing the clearance rate of the apparatus. As used herein, “in fluid communication” with a pump signifies that the pump is located along or within the fluid path, and includes configurations where no components of the pump contact the fluid, such as a peristaltic pump. A pump disposed along or within a fluid path mayor may not be in actual contact with the fluid moving along or through the path. Two plasma ports 108 are placed at either end of the plasma separator 102, one near the inlet port 104 and one near the outlet port 106, in order to provide more uniform flow through the extralumenal space. Of course, embodiments can include one, two or more plasma ports, depending on the particular application. As illustrated in the figure, the plasma ports 108 and the plasma pump 110 are configured to guide the plasma component through the extralumenal space in a direction generally perpendicular to the direction of the main flow path. Beyond the plasma pump 110, the plasma flow path ultimately reconnects with the main flow path to mix the treated plasma component with the cellular component for return to the patient.

Contaminant clearance rates in systems such as these are a function of the plasma flow rate through the binding material, the binding rate of the material, and the residence time in the binding material. For example, if the binding rate of a given material is relatively slow, then flow rates should be set accordingly so that the contaminant residence times are sufficient to allow for effective clearance. Increasing flow rates in such a situation will not effect an increase in the clearance rate, and may even result in dislodging bound toxins due to shear stresses. Thus, for a given contaminant and a given binding agent, an ideal range of plasma flow rates can be determined which optimizes the contaminant clearance rates. Thus, in some embodiments, the pump is configured to provide a plasma flow rate between 10% and 40% of the main fluid flow rate flowing into the apparatus 100 at inlet port 102. Preferably, the pump is configured to provide a plasma flow rate of approximately 25% of the fluid flow rate flowing into the apparatus 100. The plasma pump flow rate is preferably selected to increase the contaminant clearance rate by more than two times over that of a system relying on Starling flow alone, i.e., where the plasma flow is unassisted by a pump.

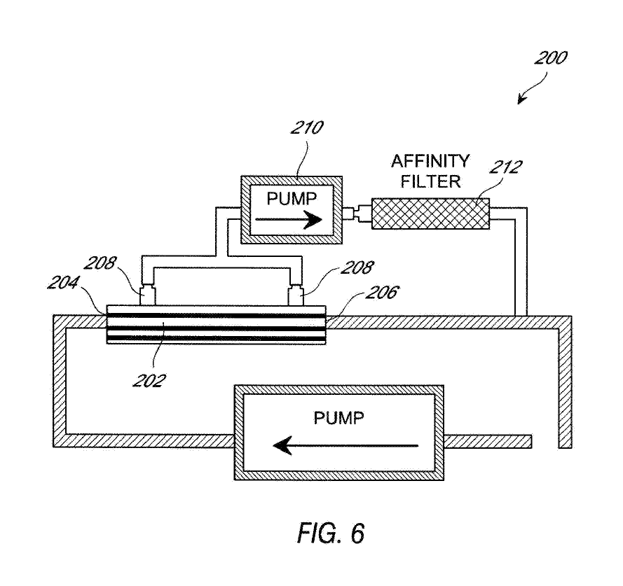

With reference to FIG. 6, an extracorporeal blood treatment apparatus 200 according to an embodiment is described. The apparatus 200 includes a plasma separator 202 having an inlet port 204, an outlet port 206, and one or more plasma ports 208 in fluid communication with a plasma pump 210. Two plasma ports 208 are placed at either end of the plasma separator 202, one near the inlet port 204 and one near the outlet port 206, as described above in connection with FIG. 5. The apparatus 200 further includes an affinity filter 212 disposed external to the plasma separator 202. The affinity filer 212 is preferably located downstream of the plasma pump 210, but can be located upstream of the pump 210.

The plasma separator 202 preferably comprises a separation membrane surrounded by a cartridge. The separation membrane has pores sized to allow passage of the plasma component of the blood across the membrane, while preventing passage of the cellular component of the blood, including blood cells and platelets. In a preferred embodiment, the separation membrane comprises one or more hollow fiber membranes as described above in connection with FIG. 5. The separation membrane functions to separate a main, e.g. blood, flow path, running from the inlet port 204 on one side of the membrane to the outlet port 206 of the plasma separator 202, from a plasma flow path, beginning on the other side of the membrane and running through the plasma port(s) 208 to the plasma pump 210. To allow contaminants to pass across the separation membrane along with the plasma, the pores can be between about 100 nm and 200 nm in diameter. In some embodiments, the pore size is between 150 and 600 nm Additionally, in some embodiments, the pores can be about, less than, less than about, more than, or more than about, 300 nm, 400 nm, 500 nm, 600 nm, 700 nm, or a range defined by any two of the aforementioned values. Preferably, the pores are of sufficient size to allow for maximization of plasma separation from platelets at the highest flow rate possible. The inlet port 204 and the outlet port 206 are in fluid communication with the main flow path, and the plasma ports 208 are in fluid communication with the plasma flow path. To withdraw fluid in an evenly distributed flow from the extralumenal space of the cartridge, the plasma ports 208 are preferably provided with wicks configured to draw fluid from inside the separator cartridge through the plasma ports 208.

The affinity filter 212 includes an affinity binding material configured to selectively bind and remove contaminants from plasma passing through the filter 212. In a preferred embodiment, the affinity filter includes immobilized lectins configured to bind glycosylated viral particles. The plasma pump 210 is configured to assist the flow of plasma traveling across the separation membrane and through the plasma ports 208 toward the affinity filter 212, thereby increasing contact with between the plasma and the affinity binding material. As illustrated in the figure, the plasma ports 208 and the plasma pump 10 are preferably disposed so as to draw the plasma component across the separation membrane in a direction generally perpendicular to the direction of the main flow path. Beyond the affinity filter 212, the plasma flow path preferably reconnects with the main flow path to mix the treated plasma component with the cellular component, in order to be returned to the patient.

In this device, the time of exposure is a function of the plasma flow rate and the capacity of the lectin-containing substrate. For example, if the whole blood flow rate of a device is 40 ml/min and the plasma assist pump is set to operate at 25% of the blood flow rate, the plasma flow rate (i.e., the assisted flow rate) is 10 ml/min. If the capacity of the lectin-containing substrate is 10 ml, then running unprocessed blood at 40 ml/min (that is, running plasma at 10 ml/min) for 30 minutes would process 1200 ml of blood, exposing 300 ml of plasma to the lectin-containing substrate, each ml exposed for 1 minute. If, instead of continuously processing blood, a blood pool volume of 120 ml were recirculated through the same device for 30 minutes, then 30 ml of plasma would be exposed to the contaminant-binding substrate, each ml exposed for 10 minutes. In some embodiments, the time the plasma is exposed to the lectin-containing substrate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 minutes. In other embodiments, the time the plasma is exposed to the lectin-containing substrate is a range defined by any two times recited above.

In another embodiment, the blood flow rate into the device is about 20 ml/min to about 500 ml/min. In another preferred embodiment, the blood flow rate into the device is about 250 ml/min to about 400 ml/min. In some embodiments, the blood flow rate is, is about, is less than, is less than about, is more than, is more than about, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml/min, or a range defined by any two of these values. In some embodiments, the plasma flow rate is, is about, is less than, is less than about, is more than, is more than about, 10%, 12%, 14%, 16%, 18%, 20%, 22%, 24%, 26%, 28%, 30%, 32%, 34%, 36%, 38%, 40%, 45%, 50%, 55%, 60%, 65%, or 70% of the blood flow rate, or a range defined by any two of these values. In some embodiments, the capacity of the device is 40 ml. Also contemplated are devices where the capacity is about, is less than, is less than about, is more than, is more than about, 3000, 2000, 1500, 1000, 750, 600, 550, 500, 490, 480, 470, 460, 450, 440, 430, 420, 410, 400, 390, 380, 370, 360, 350, 340, 330, 320, 310, 200, 290, 280, 270, 260, 250, 240, 230, 220, 210, 200, 190, 180, 170, 160, 150, 140, 130, 120, 110, 100, 90, 80, 70, 60, 50, 40, 30, 20, 19, 18, 17, 16, 15, 14, 13, 12, 11, 10, 9, 8, 7, 6, 5, 4, 3, 2, or 1 ml, or a range defined by any two of these values.

Methods for treating the blood of individuals infected with contaminants are also described. Whole blood can be collected from an infected individual and supplied to a separator means configured to separate the whole blood into a cellular component and a plasma component. The separator means preferably comprises a hollow fiber membrane contained within a cartridge; however, embodiments can also include other types of separator means known in the art, such as a centrifuge, for example. The plasma component is passed through a contaminant affinity medium, such as, for example, a lectin-containing affinity matrix, which is disposed within the separator cartridge or in an external affinity cartridge. The flow rate of the plasma component through the separator, and through the affinity-binding medium, is preferably augmented by a plasma pump disposed external to the separator. The plasma is pumped at an assisted flow rate between 5% and 70%, preferably between 10% and 40%, of the whole blood flow rate. The assisted flow rate is selected to provide a contaminant clearance rate effective to reduce viral load in the infected blood. For example, where the virus has a replication rate of over 10¹¹ viral copies per day, the assisted flow rate can be selected to provide a T_{90%} in under 1 hour. In other embodiments, the assisted flow rate is selected such that the clearance rate of the assisted flow device relative to the same or substantially similar device without assisted flow is, is about, is greater than, is greater than about, 1.25, 1.50, 1.75, 2.0, 2.25, 2.50, 2.75, 3.0, 3.5, 4.0, 4.5, 5.0, 5.5, 6.0, 6.5, 7, 8, 9, 10, 11, 12, 13, 14, 15, 20, 25, 30, 35, 40, 45, or 50, or a range defined by any two of these values. In another embodiment, the assisted flow rate is selected such that the T_{90%} is reduced compared to the same or substantially similar unassisted device by, by about, by at least, by at least about, 5, 10, 15, 20, 25, 30, 35, 40, 45, 50, 55, 60, 65, 70, 75, 80, 85, 90, 95 or 99%, or a range defined by any two of these values. In other embodiments, the assisted flow device is configured such that a log plot of the percentage contaminant remaining versus time is linear, or approximately linear from 100% contaminant remaining to a value of percent remaining of, of about, of less than, of less than about, 40, 35, 30, 25, 20, 15, 14, 13, 12, 11, 10, 9, 8, 7, 6, 5, 4, 3, 2, or 1%, or a range defined by any two of these values. After the plasma component has passed through the affinity-binding medium, the treated plasma can be mixed with the cellular component and ultimately be returned to the patient, or stored separately.

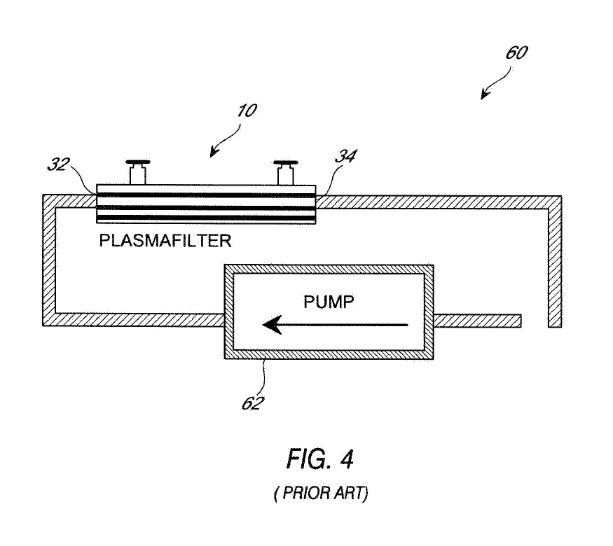

With reference to FIGS. 1-4, a conventional system 60 is illustrated which utilizes the above-described plasmapheresis device 10. Whole blood is withdrawn from a subject or other source using a pump 62, and pumped into the inlet port 32 of the device 10. As blood flows through the device 10, plasma filters through the membrane 42 and into the exterior portion 44 by convective flow, also known as Starling flow. High pressure at the proximal inlet port 32 of the device 10 forces plasma through pores in the membrane 42, allowing the plasma to contact the lectins 46 in the exterior portion 44. Blood cells and platelets are too large to pass through the pores in the membrane 42, and remain in the lumen of the hollow fibers. At the distal outlet port 34 of the cartridge, reduced luminal pressure allows the treated plasma to return to the lumen and thus to the blood as it exits the device 10. In some embodiments, the main blood flow pump 62 is downstream of the device 10.

In another embodiment, the device comprises a processing chamber having lectin disposed within the processing chamber, wherein said lectin binds viral particles or fragments in the blood or plasma, and traps them in the processing chamber. The blood or plasma can directly contact the lectins. In other embodiments, the device has a porous membrane which divides the chamber into one or more portions, such that the lectin is located in only a portion of the chamber. The preferred device utilizes hollow channel fiber membranes, but one or more sheets of membranes that divide the chamber are also contemplated. Where a membrane is used, the blood or plasma is filtered by the membrane, such that some portion of the blood or plasma is excluded from the portion of the chamber containing the lectin (e.g., blood cells or other large cells which cannot pass through the pores of the membrane).

In systems such as these, the cartridge is sealed, and relies on convective flow, or Starling flow, to drive plasma into contact with the affinity-binding agent. Unfortunately, the magnitude of Starling flow across a membrane can be relatively low for high viscosity fluids like plasma, as compared to the total fluid flow into the device. Direct measurements indicate that plasma flow rate is less than 10%, and often less than 8%, of the total blood flow rate. During use, blood elements can accumulate near and possibly clot or clog the pores, further reducing the plasma flow rate and, as a result, reducing the clearance rate.

In another embodiment, the lectin affinity hemodialysis device disclosed herein utilizes a pump to increase the plasma flow rate, relative to the whole blood flow rate, in order to improve plasma contact with the affinity-binding agent. The pump assists plasma flow through a separation membrane and/or through an affinity material. In some embodiments, the affinity binding material is disposed proximate to the separation membrane, within a single separation cartridge. In other embodiments, the affinity binding material is disposed external to the separation cartridge. These and other embodiments advantageously provide contaminant clearance rates that are preferably at least two times faster than those of conventional systems, without effecting a significant change in hemolysis rates. Thus, embodiments can be used to effectively reduce viral load in patients infected with rapidly replicating viruses, such as HCV or Dengue hemorrhagic fever virus. Embodiments can also be used to provide a more rapid and efficient clearance of slower-replicating viruses such as HIV.

In some embodiments, a lectin affinity hemodialysis device and method for reducing the viral load or pfu/ml in the blood or plasma by a therapeutically effective amount are provided. As used herein, the term “therapeutically effective amount” refers to a viral load or pfu/ml in the blood or plasma that halts or slows the progression of the infection, and slows and prevents the worsening of symptoms associated with the infection, and preferably improves and eliminates the infection or symptoms thereof. In some cases, reducing viral load or pfu/ml by or to a “therapeutically effective amount: can allow an infected individual's immune system to maintain or reduce the viral load or pfu/ml without further intervention. In some embodiments, “therapeutically effective amount” is an amount sufficient to render another treatment (e.g. a drugs, retroviral therapy, etc.) effective, or more effective. The “therapeutically effective amount” can vary with different viruses and individuals, but can be readily determined by a skilled artisan.

As evidenced by Table 1 below, the copies of virus per ml, varies from virus to virus. Just as the average viremia before clearance varies between viruses, so does the desired viral load or pfu/ml after clearance. In some embodiments, a “therapeutically effective amount,” or the desired viral load or pfu/ml after clearance is, is about, is less than, is less than about, is more than, is more than about 1×10⁹, 5×10⁸, 1×10⁸, 5×10⁷, 1×10⁷, 5×10⁶, 1×10⁶, 500,000, 450,000, 400,000, 350,000, 300,000, 250,000, 200,000, 150,000, 100,000, 90,000, 80,000, 70,000, 60,000, 50,000, 45,000, 40,000, 35,000, 30,000, 25,000, 20,000, 15,000, 10,000, 9000, 8000, 7000, 6000, 5000, 4000, 3000, 2000, 1000, 900, 800, 700, 600, 500, 450, 400, 350, 300, 250, 200, 190, 180, 170, 160, 150, 140, 130, 120, 100, 95, 90, 85, 80, 75, 70, 65, 60, 55, 50, 45, 40, 35, 30, 25, 20, 15, 10, 0. In some embodiments, the desired pfu/ml after clearance is a range defined by any two of the preceding numbers.

TABLE 1
Human Viral Infections

| | Viremia (copies per ml plasma) ^{a} | | | | Preference |
|---|---|---|---|---|---|
| Viruses | Max | Mean | Survivable | Lethal | (number of patients) |
| Crimean Congo hemorrhaqic | | 7.7 × 10^{5 (1)} | | | 1 (n = 1) |
| fever | | | | | |
| Dengue fever | 1.5 × 10^{7 (5)} | 4.0 × 10^{7 (11)} | 4.0 × 10^{7 (11)} | | 5 (n = 20), 11 (n = 31) |
| | | 8 × 10⁵ | | | 1 (n = 1) |
| febrile | | 1.2 × 10^{5 (5)} | | | 5 (n = 20) |
| defevrescent | | Not | | | 5 (n = 20) |
| | | detectable | | | |
| Dengue hemorrhagic fever | 2.0 × 10⁹ | 3.2 × 10^{8 (11)} | 4.0 × 10⁷ | 3.2 × 10^{8 (11)} | 11 (n = 31) |
| febrile | | 1.5 × 10^{6 (5)} | | | |
| defevrescent | | 4.3 × 10^{5 (5)} | | | |
| Ebola | 1 × 10^{9 (7)} | | 1 × 10^{7 (7)} | 6.9 × 10^{8 (1)} | 1, 7 (n = 3) |
| Hepatitis C virus | | 3.2 × 10⁶ | | | National Genetic Inst |
| HIV | 2 × 10^{6 (15)} | 2 × 10^{4 (15)} | 1 × 10^{3 (16)} | | 15 (n~100) |
| Influenza | not done | | | | |
| Lassa virus | 4 × 10^{9 (1)} | 7 × 10^{6 (1)} | | 4 × 10^{3 (8)} | 1, 8 (n = 46) |
| | 1.0 × 10^{9 (9)} | | | | 9 (n = 2) |
| Rift Valley fever | 1.0 × 10^{9 (13)} | | | | 13 |
| Sin Nombre | | 1.3 × 10^{6 (4)} | 6.3 × 10^{5 (4)} | 5.0 × 10^{6 (4)} | 4 (n = 26) |
| Smallpox (Vaccinia) | 2 × 10^{5 (12)} | | | | 12 (n = 10) |
| West Nile Virus | 1 × 10^{7 (10)} | | | | 10 (n = 1) |
| Yellow fever | 1 × 10^{6 (14)} | 4 × 10^{5 (1)} | | | 1 (n = 1) |

^{a} Viral load in copies per ml plasma is shown in scientific notation followed by the specific reference in parenthesis

### REFERENCES

1. Gunther, S (2002) J. Clinical Microbiology, 40 (7):2323-2330.
2. Drosten, et al NEJM 348 (20):1967-76, 2003
3. Zwiers, Miller, Baker, Kulesh, Jahrling and Huggins (USAMRIID)
4. Terajima, et al (1999) J Infect Dis 180:2030.
5. Wang, W K et al (2003) Virology 20:330.
6. Sanchez, et al (2004) J, Virol 18:10370
7. Towner et al (2004) J. Virol 78:4330
8. McCormick et al (1986) NEJM 314:20
9. Schmitz et al (2002) Microbes Infect 4(1):43-50
10. Paddock et al. (2006) CID 2006:42 (June 1) 1527
11. Vaughn et al (2000) J Infect Dis 181:2-9
12. Sharon et al (2003) JAMA Jun. 25 2003 289 (24) 3295
13. Niklasson et al (1983) Journal of Clinical Microbiology 1026-1031 17(6)
14. Monath et al (2001) Lancet Infectious Diseases 1: 11-20

In one embodiment, the lectin affinity hemodialysis device is attached to an individual wherein the inlet port of the device is linked to the individual's vascular system, allowing blood to flow from the individual into the device, optionally with the assistance of a pump. In other embodiments, the blood from the individual is filtered or separated, allowing only the virus containing component to be exposed to a lectin-containing membrane. In some embodiments, the outlet port is also linked intravenously to the individual to allow the effluent blood to be reinfused into the individual. In one embodiment, the purified plasma is mixed with the cellular component before being reinfused into the individual. In another embodiment, the cellular component of the blood is reinfused into the individual separate from the effluent plasma.

In some embodiments, a volume equal to the total blood volume of the individual being treated is allowed to circulate at least once through the device. This does not necessarily mean that all of the blood in the individual passes through the device. As the blood is filtered and recirculated into the individual's blood stream, it is diluted by blood already present in the individual's blood stream. As such, it would be difficult to determine when all of the blood in the individual is circulated through the device. However, it can be determined when a volume equal to all of the individual's blood has been treated. Accordingly, the volume equal to the total blood volume of the individual being treated is defined as the total volume of blood run through the device being approximately equal to the estimated total blood volume present in the bloodstream of the individual being treated. For humans, the total blood volume for an average adult male weighing approximately 70 kg is between approximately 4 L and 5 L, (approximately 66 ml/kg) and the total volume of blood for an average adult female weighing approximately 50 kg is between approximately 3.0 L and 3.5 L (approximately 60 ml/kg). In some embodiments, a multiple of the total blood volume is treated. This multiple is, is about, is less than, is less than about, is more than, is more than about, 0.5, 1, 1.5, 2, 3, 4, 5, 6, 7, 8, 9, 10, 15, 20, 30, 40, 50, 60, 70, 80, 90, or 100, or a range defined by any two of these amounts.

The number of times the volume of blood being treated is required to be circulated through the device (treatment cycles) varies based on the replication rate of the virus being treated, the viral load or pfu/ml of the individual's blood, and the clearing rate of the device. The replication rate of viruses varies with each virus, but is known or can be determined by one skilled in the art. The viral load or pfu/ml within the individual's blood is dictated by the replication rate of the virus less the clearance rate of the virus. Further, the percentage of virus within the organs (non-blood borne), and the level of infectivity of the individual being treated influence the viral load, but can be ascertainable by a skilled artisan. The clearing rate of a particular device, although usually fixed across a broad spectrum of viruses, can vary. The clearing rate of a particular device is ascertainable by a person of ordinary skill in the art. Accordingly, the clinically relevant number of circulations is ascertainable without undue experimentation. The term “therapeutically effective number of circulations,” as used herein, refers to the number of circulations determined by a person of ordinary skill in the art to reduce the pfu/ml or viral load of the blood by or to a therapeutically effective amount.

In some embodiments, the number of times the blood or plasma being treated, which can be equal to the total blood volume of the individual being treated, or a multiple thereof, circulates through the device is, is about, is less than, is less than about, is more than, is more than about 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, 1. In some embodiments, the number of times the volume of blood equal to the total blood volume of the individual being treated circulates through the device is a range defined by any two numbers recited above.

Once the amount of blood or plasma to be processed and the number of circulations is determined, the time required for lectin affinity hemodialysis treatment is determined by the flow rate and capacity of the device. As such, the time required for a volume of blood or plasma to be processed on the device, or the amount of time an individual is treated by the device, can be determined by a skilled artisan. In some embodiments, the time required is, is about, is less than, is less than about, is more than, is more than about 600, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 minutes. In other embodiments, the time required for an individual to be processed on the device is a range defined by any two times recited above. In some embodiments, the individual's blood is continuously treated, and the device, or lectin portion of the device is periodically replaced.

In some embodiments, the lectin affinity hemodialysis treatment reduces the viral load or pfu/ml in the blood or plasma by, by about, by at least, by at least about, by more than, by more than about 99.9, 99.8, 99.5, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 45, 40, 35, 30, 25, 20, 15, or 10%. In other embodiments, the process reduces the viral load in the blood or plasma by a range defined by any two percentages recited above.

In some embodiments, the reduction in viral load or pfu/ml occurs within a limited amount of time. The amount of time required to reduce the viral load or pfu/ml to a clinically relevant viral load, or by a certain amount, is, is about, is less than, is less than about, is more than, is more than about 50, 45, 40, 35, 30, 25, 20, 15, 10, 5, 4, 3, 2 or 1 days, 23, 22, 21, 20, 19, 18, 17, 16, 15, 14, 13, 12 or 11 hours, 600, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 minutes.

In some embodiments, the lectin affinity hemodialysis devices and methods of the present disclosure preferentially remove live viral particles (pfu) from blood or plasma more readily than other viral particles or fragments thereof. In some embodiments, the ratio of percent pfu clearance to percent viral load clearance is, is about, is less than, is less than about, is more than, is more than about, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.5:1, 7.0:1, 7.5:1, 8.0:1, 8.5:1, 9.0:1, 9.5:1, 10:1, 15:1, 20:1, 30:1, 40:1, 50:1, 75:1, 100:1, 125:1, 150:1, 175:1, or 200:1. In other embodiments, the ratio of pfu clearance to viral load clearance is a range defined by any two ratios recited above.

In one embodiment, blood having viral particles and/or fragments thereof is withdrawn from a patient and contacted with a membrane. In one preferred embodiment, the blood is separated into its plasma and cellular components. The plasma is then contacted with the lectins to remove the viral particles or fragments thereof by binding between viral high mannose glycoproteins and lectins. The plasma can then be recombined with the cellular components and returned to the patient. Alternatively, the cellular components can be returned to the patient separately. The treatment can be repeated periodically until a desired response has been achieved.

The technology to immobilize enzymes, chelators, and antibodies in dialysis-like cartridges has been developed (Ambrus et al., Science 201(4358): 837-839, 1978; Ambrus et al., Ann Intern Med 106(4): 531-537, 1987; Kalghatgi et al. Res Commun Chem Pathol Pharmacol 27(3): 551-561, 1980) and is incorporated herein by reference. These cartridges can be directly perfused with blood from patients through direct venous access, and returned to the patients without further manipulations. Alternatively, blood can be separated into plasma and cellular components by standard techniques. The cellular components can be combined with the plasma before reinfusing or the cellular components can be reinfused separately. Viral load can be assessed in the effluent from the cartridge by standard techniques such as ELISA and nucleic acid amplification and detection techniques. Prototypic cartridges have been used to metabolize excess phenylalanine (Kalghatgi et al., 1980, supra; Ambrus, 1978, supra) or to remove excess aluminum from patients' blood (Anthone et al. J Amer Soc Nephrol 6: 1271-1277, 1995). An illustration of preparing proteins for immobilization to the hollow fibers for the method of the present disclosure is presented in U.S. Pat. Nos. 4,714,556 and 4,787,974, 5,528,057 which are incorporated by reference herein by their entirety.

For binding of lectins to the membrane, the polymers of the membrane are first activated, i.e., made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3). Once the polymer has been activated, the lectins can be attached directly or via a linker to form in either case an affinity matrix. Suitable linkers include, but are not limited to, avidin, strepavidin, biotin, protein A, and protein G. The lectins can also be directly bound to the polymer of the membrane using coupling agents such as bifunctional reagents, or can be indirectly bound. In a preferred embodiment, GNA covalently coupled to agarose can be used to form an affinity matrix.

In some embodiments, the lectin is attached to a substrate instead of, or in addition to, the membrane. Suitable substrates include, but are not limited to, silica (e.g. glass beads, sand, diatomaceous earth) polysaccharides (e.g. dextran, cellulose, agarose), proteins (e.g. gelatin) and plastics (e.g. polystyrenes, polysuflones, polyethersulfones, polyesters, polyurethanes, polyacrylates and their activated and native amino and carboxyl derivatives). The lectin can be bound to the substrates through standard chemical means, either directly, or through linkers such as C2 to C>20 linear and branched carbon chains, as well as the plastics, proteins and polysaccharides listed above. For most synthetic purposes, C18 is the preferred upper limit but the chains can be added together for solubility reasons. Preferred linkers include: C2 to C18 dicarboxylates, diamines, dialdehydes, dihalides, and mixtures thereof (e.g. aminocarboxylates) in both native and activated form (e.g. disuccinimidyl suberimidate (DSS)). In some embodiments, one or more substrates can be used as linkers, alone or in combination with the substances listed as linkers. For example, dextran can be attached to sand, and additional linkers can then optionally be added to the dextran.

As used herein, individual or subject, refers to any animal whose blood or other bodily fluid is being treated, and is not limited to humans. Individuals or subjects include all animals, including but not limited to primates such as monkeys and apes, dogs, cats, rats, mice, rabbits, pigs, and horses.

Although illustrated within the context of a lectin-based binding medium for removing glycosylated viral particles, embodiments of the present disclosure can also be used with any other contaminant-removing plasmapheresis system for which increased clearance rates and efficiency are desirable. For example, embodiments can be used with plasmapheresis systems comprising other binding materials for removing contaminants, such as activated charcoal as a binding agent for removing chemotherapeutic agents. It will be understood by those skill in the art that numerous and various modifications can be made without departing from the spirit of the present disclosure. Therefore, it should be clearly understood that the forms of the methods and devices described herein are illustrative only and are not intended to limit the scope of the invention.

Although the embodiments described herein refer to removal of virus particles or fragments thereof from blood or plasma, one of skill in the art will appreciate that the device and methods described herein can be used with other fluids, such as other bodily fluids, cell culture supernatants, buffers, etc., which are contaminated with or contain lectin—binding virus or viral particles.

U.S. patent application Ser. No. 10/760,810, issued as U.S. Pat. No. 7,226,429, and the articles, patents, and other printed materials referred to herein, are hereby incorporated by reference in their entirety, and particularly for the material referred to above.

The Aethlon Hemopurifier® has proven effective in capturing the reconstructed Spanish Flu of 1918 virus (1918rv). During in vitro testing, high concentrations of 1918rv were rapidly depleted from cell culture fluid when circulated through the Hemopurifier®. The study documented that 76 percent of 1918rv was removed from circulation during the first two hours, and by the end of the sixth hour, 93 percent of 1918rv was cleared from circulation. The study data was quantified by reverse transcription polymerase chain reaction (PCR). The Spanish Flu of 1918 is believed to have caused 40-50 million deaths during a two-year time span. The Hemopurifier® has also demonstrated effectiveness in capturing H5N1 avian influenza (bird flu). As previously disclosed, the Hemopurifier® removed up to 99.4 percent of infectious H5N1 virus from cell culture fluids during a six-hour testing period. Scientists are increasingly worried that H5N1 avian flu could mutate into a strain that triggers a global pandemic that rivals the Spanish Flu of 1918.

These data demonstrating effectiveness against history's most lethal form of influenza and the looming H5N1 threat validates the Hemopurifier® as an innovative strategy to address current and future pandemic flu threats. The data is especially timely, as scientists have discovered that H5N1 virus has emerged to be resistant to the globally stockpiled drug Tamiflu.

The May 15, 2008 issue of the science journal Nature reported that researchers have confirmed that H5N1 avian influenza virus has mutated to become resistant to Tamiflu. As per the recommendation of the World Health Organization (WHO), Tamiflu is an antiviral drug agent that has been stockpiled by governments around the world as a potential treatment against pandemic influenza. Researchers now believe that viral mutation will necessitate a multi-pronged treatment approach against future flu pandemics, as antiviral drugs are unlikely to provide clinical benefit as stand-alone therapies. The Hemopurifier® can enhance the benefit of stockpiled drugs and future candidate therapies by clearing the viral strains from circulation that cause drug and vaccine resistance.

The Hemopurifier® is a broad-spectrum therapeutic device able to separate and then capture circulating viruses by glycoproteins that reside on their surface. In the case of pandemic influenza, the Hemopurifier® is able to separate and then capture circulating influenza virus by hemagglutinin (HA) and neuraminidase (NA) glycoproteins that reside on the virus surface regardless of mutation. As a result, the applications of the Hemopurifier® against pandemic influenza include:

The Hemopurifier® is a first-line countermeasure against highly virulent strains of pandemic influenza that are untreatable with drug and vaccine therapies.

The Hemopurifier® assists in the initial identification of emerging influenza strains through the concentration and capture of pandemic influenza viruses from the entire circulatory system of infected patients, thus directing the development of candidate drug and vaccine therapies towards the strain of influenza virus that sparks widespread infection.

The Hemopurifier® may also address the needs of immunocompromised and at risk populations, including children, pregnant women, and senior citizens for whom the administration of drugs or vaccines developed against pandemic influenza may be medically contraindicated.

Influenza A is primarily a respiratory virus with a low level of lethality and little indication of transmission via the blood. However, certain strains of the virus, such as H5N1 bird flu, H1N1 swine flu, and the 1918 Spanish flu, have greatly increased mortality and morbidity. For these there is significant indication of blood borne viremia that can transmit the virus to other vital organs (de Jong, M, et al. N.E.J. Med 2006. 352:686; Zou, 2006 Transfus Med Rev 20(3):181-189). For these types of influenza infections, the enhanced antiviral therapy methods disclosed herein which use an antiviral therapy in combination with a lectin affinity hemodialysis treatment would be efficacious. The enhanced antiviral therapy methods disclosed herein can be used for the removal of any blood-borne viruses to which lectins bind. For example, viruses which can be cleared by the enhanced antiviral therapy methods disclosed herein include, but are not limited to, enveloped virus, Category A enveloped virus, ebola, marburg, smallpox, lassa, dengue, rift valley, west nile, influenza (e.g., H5N1 and H1N1), measles, mumps, viral encephalitis (e.g. Japanese encephalitis), monkeypox, camelpox, vaccinia, HIV, HCV, hepatitis virus, human cytomegalovirus (HCMV), swine pox, swine flu, siv, fiv, bird flu, sin nombre, yellow fever, herpes, SARS, sendai. In other embodiments, one or more viruses from the families of retroviridae, poxviridae paramyxoviridae (e.g., measles, mumps, sendai), orthomyxoviridae (e.g., bird flu, influenza), filoviridae (e.g., ebola, marburg), coronaviridae (e.g., SARS, encephalomyelitis), herpesviridae (e.g., herpes simplex, HCMV), rhabdoviridae (e.g., varicella stomatitis, rabies), and togavirus (e.g., rubella, semliki), are cleared. As used herein, “lectin-binding virus” is a virus which binds to or is bound by lectin. In some embodiments, the virus is not HIV or HCV.

In one embodiment, the enhanced antiviral therapy methods disclosed herein are used as a broad-spectrum treatment against bioterror threats. Smallpox is considered to be a Category “A” bioterror threat by the National Institute of Allergy and Infectious Diseases (NIAID). As research with human infectious smallpox is prohibited, MPV represents a primary model to study candidate therapies for smallpox virus. In one embodiment, concentrations of MPV are rapidly depleted from the blood or plasma of an infected individual.

Vaccinia is the “live pox-type virus” used in the smallpox vaccine. In one embodiment, high concentrations of vaccinia virus are rapidly depleted from the blood or plasma of an infected individual.

The reconstructed 1918 influenza virus tested in Hemopurifier® in vitro studies, and depicted as 1918rv in this disclosure, was a recombinant virus with two genes (the HA and NA) from the 1918 strain of influenza along with six genes from the Texas 91 influenza strain. The resulting research virus is known as 1918 HA/NA:TX/36/91 in scientific literature. It is anticipated that the use of the Hemopurifier® will be directed towards pandemic strains of influenza whose virulence is attributed by survival and spread in the circulatory system of infected patients.

In some embodiments, the exact regimen of a lectin affinity hemolysis treatment is determined on a patient-by-patient basis, in many cases, some generalizations regarding the regime can be made. In some embodiments, the lectin affinity hemodialysis treatment for a human patient is administered 1 to 4 times a day. As will be understood by those of skill in the art, in certain situations it may be necessary to administer the hemodialysis treatment disclosed herein in frequencies that exceed, or even far exceed, the above-stated, preferred dosage range in order to effectively and aggressively treat particularly aggressive diseases or infections. The lectin affinity hemodialysis treatment may be administered for a continuous period, for example for a week or more, or for months or years. In some embodiments, the lectin affinity hemodialysis treatment is administered for a period of time, which time period can be, for example, from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 42, 45, 49, 50, 55, 56, 60, 63, 65, 70, 75, 77, 80, 84, 85, 90, 91, 95, 98, 100, 105, 110, 112, 115, 119, 120, 125, 126, 130, 133, 135, 140, 145, 147, 150, 154, 155, 160, 165, 170, 175, 180, 185, 190, 195, 200, 205, 210, 215, 220, 225, 230, 235, 240, 245, 250, 260, 270, 280, 290, 300, 310, 320, 330, 340, 350, or 360 days, or 12, 13, 14, 15, 16, 17, 18, 19, 20, 21, 22, 23, 24 months, or longer. In some embodiments, the regimen of the lectin affinity viral hemodialysis treatment is administered four times a day, three times a day, twice a day, once a day, every other day, three times a week, every other week, three times per month, once monthly, substantially continuously or continuously.

The particular features described in the embodiments above are not limited to the embodiments in which they are described, but can be combined with any of the embodiments of the disclosed methods and devices. The following examples are presented to illustrate embodiments of the present disclosure and are not intended to be restrictive.

### EXAMPLE 1

A patient suffering from HCV infection is identified. A lectin affinity hemodialysis treatment is administered to the patient for 8 hours a day, 3 times a week for a week prior to administering the patient a course of interferon and ribavinrin (IFN/RIB) combination therapy. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

After the completion of the one-week hemodialysis treatment, a course of the IFN/RIB combination therapy is administered to the patient for 24 weeks. During the courses of hemodialysis and IFN/RIB treatment, the viral load is monitored by quantifying HCV-RNA using the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces at a higher rate as compared to patients that receive either the lectin affinity hemodialysis treatment or the IFN/RIB therapy alone. The patient achieves a sustained viral response (SVR) in a shorter amount of time compared to patients that receive either the hemodialysis treatment or the IFN/RIB therapy alone. Accordingly, the use of the lectin affinity hemodialysis treatment prior to the antiviral therapy improves the efficacy of the treatment as compared to either the lectin affinity hemodialysis treatment or the IFN/RIB therapy alone. Preferably, the improvement in the efficacy of the treatment is additive; more preferably, the improvement in the efficacy of the treatment is synergistic.

### EXAMPLE 2

A patient suffering from HCV infection is identified. A lectin affinity hemodialysis treatment is administered to the patient for 8 hours a day, 3 times a week for 12 weeks. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

After one week of the lectin affinity hemodialysis treatment, a course of the IFN/RIB combination therapy is administered to the patient for 24 weeks. During the courses of hemodialysis and IFN/RIB treatment, the viral load is monitored by quantifying HCV-RNA using the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces at a higher rate as compared to patients that receive either the lectin affinity hemodialysis treatment or the IFN/RIB therapy alone. The patient achieves a sustained viral response (SVR) in a shorter amount of time compared to patients that receive either the hemodialysis treatment or the IFN/RIB therapy alone. Accordingly, the use of the lectin affinity hemodialysis treatment prior to and/or in combination with the antiviral therapy improves the efficacy of the treatment as compared to either the lectin affinity hemodialysis treatment or the IFN/RIB therapy alone. Preferably, the improvement in the efficacy of the treatment is additive; more preferably, the improvement in the efficacy of the treatment is synergistic.

### EXAMPLE 3

A patient suffering from HCV infection is identified. A lectin affinity hemodialysis treatment is administered to the patient for 8 hours a day, 3 times a week for 24 weeks. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

From the same day on which the lectin affinity hemodialysis treatment starts, a course of the IFN/RIB therapy is administered to the patient for 24 weeks. During the courses of hemodialysis and IFN/RIB treatment, the viral load is monitored by quantifying HCV-RNA using the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces at a higher rate as compared to patients that receive either the lectin affinity hemodialysis treatment or the IFN/RIB therapy alone. The patient achieves a sustained viral response (SVR) in a shorter amount of time compared to patients that receive either the hemodialysis treatment or the IFN/RIB therapy alone. Accordingly, the use of the lectin affinity hemodialysis treatment in combination with the antiviral therapy improves the efficacy of the treatment as compared to either the lectin affinity hemodialysis treatment or the IFN/RIB therapy alone. Preferably, the improvement in the efficacy of the treatment is additive; more preferably, the improvement in the efficacy of the treatment is synergistic.

### EXAMPLE 4

A patient suffering from HCV infection is identified. A lectin affinity hemodialysis treatment is administered to the patient for 8 hours a day, 3 times a week for 1 week. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

From the same day on which the lectin affinity hemodialysis treatment starts, a course of the IFN/RIB therapy is administered to the patient for 46 weeks. On day 1 of the lectin affinity hemodialysis treatment, IFN is administered intramuscularly to the patient 1 hour after the completion of the lectin affinity hemodialysis treatment. During the courses of hemodialysis and IFN/RIB treatment, the viral load is monitored by quantifying HCV-RNA using the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces at a higher rate as compared to patients that receive either the lectin affinity hemodialysis treatment or the IFN/RIB therapy alone. The patient achieves a sustained viral response (SVR) in a shorter amount of time compared to patients that receive either the hemodialysis treatment or the IFN/RIB therapy alone. Accordingly, the use of the lectin affinity hemodialysis treatment in combination with the antiviral therapy improves the efficacy of the treatment as compared to either the lectin affinity hemodialysis treatment or the IFN/RIB therapy alone. Preferably, the improvement in the efficacy of the treatment is additive; more preferably, the improvement in the efficacy of the treatment is synergistic.

### EXAMPLE 5

A patient suffering from HCV infection is identified. The patient is administered interferon (IFN) and ribavirin (RIB). When it is found that the patient stops responding to the therapy, a lectin affinity hemodialysis treatment is administered to the patient for 8 hours a day, 3 times a week for 12 weeks, while the IFN/RIB therapy is continued to be administered to the patient concurrently with the hemodialysis treatment for 12 weeks. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

During the courses of hemodialysis treatment and IFN/RIB treatment, the viral load is monitored by quantifying HCV-RNA through the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces significantly compared to the administration of the IFN/RIB therapy alone, and the patient achieves a sustained viral response (SVR) after 12 weeks of lectin affinity hemodialysis treatment.

### EXAMPLE 6

A patient suffering from HCV infection is identified. The patient is administered a therapy of interferon (IFN) and ribavirin (RIB). The IFN/RIB therapy is ended when it is found that the patient stops responding to the therapy after 6 weeks. After the completion of IFN/RIB therapy, a lectin affinity hemodialysis treatment is administered to the patient for 8 hours a day, 3 times a week for 12 weeks. During the hemodialysis treatment, the inlet port of a lectin affinity hemodialysis device is linked intravenously to the patient to allow blood to flow from the patient to the device, optionally with the assistance of a pump. Blood is collected from a peripheral vein of the patient to pass through the lectin affinity hemodialysis device. The outlet of the lectin affinity hemodialysis device is also linked intravenously to the patient to allow the effluent blood to be reinfused into the patient.

During the courses of hemodialysis treatment and IFN/RIB treatment, the viral load is monitored by quantifying HCV-RNA through the original Amplicore HCV monitor method at various time points. The quantity for viral response rate is measured by the qualitative Amplicore HCV monitor (detection limit: 0.05 KIU/ml), and any quantity below the detection limit is taken as to be negative.

The viral load of the patient reduces significantly compared to the administration of the IFN/RIB therapy alone, and the patient achieves a sustained viral response (SVR) after 12 weeks of lectin affinity hemodialysis treatment.

### EXAMPLE 7

A patient suffering from HIV infection is identified. The patient is treated and the efficacy of the enhanced antiviral therapy is measured according to the procedures disclosed in Examples 1-6, except that the viral infection is HIV infection and the antiviral therapy is the administration of the Highly Active Antiretroviral Therapy (HAART) to the patient. The HAART therapy comprises at least three anti-HIV drugs selected from entry inhibitors (e.g., Fuzeon® and Selzentry™), nucleoside reverse transcriptase inhibitors (NRTIs, e.g., Atripla® and COMBIVIR®), non-nucleoside reverse transcriptase inhibitors (NNRTIs, e.g., Atripla® and Rescriptor®), integrase inhibitors (e.g., Isentress), and protease inhibitors (PIs, e.g., Crixivan® and Viracept®)

### EXAMPLE 8

A patient suffering from infection by a lectin-binding virus is identified. The patient is treated and the efficacy of the enhanced antiviral therapy is measured according to the procedures disclosed in Examples 1-6, except that the virus is a lectin-binding virus and the antiviral therapy is the standard antiviral therapy specifically designed for that lectin-binding virus.

All references mentioned herein are hereby incorporated by reference in their entireties and for the material specifically referenced herein. From the foregoing, it will be obvious to those skilled in the art the various modifications in the above-described methods, devices and compositions can be made without departing from the spirit and scope of the present disclosure. Accordingly, the disclosed methods and devices herein can be embodied in other specific forms without departing from the spirit or essential characteristics thereof. Present examples and embodiments, therefore, are to be considered in all respects as illustrative and not restrictive, and all changes which come within the meaning and range of equivalency of the claims are therefore intended to be embraced therein.

1-53. (canceled)

54. An enhanced antiviral therapy method for treating an individual suffering from viral infection, the method comprising:
administering to said individual a course of antiviral therapy; and
enhancing the efficacy of said antiviral therapy by administering a lectin affinity hemodialysis treatment to said individual, wherein said lectin affinity hemodialysis treatment comprises passing blood or plasma from said individual through a lectin affinity hemodialysis device, wherein a lectin in said lectin affinity hemodialysis device binds a virus or fragments thereof in said blood or plasma, and wherein said lectin traps said virus or fragments thereof in said lectin affinity hemodialysis device, removing said virus or fragments thereof from said blood or plasma.

55. The method of claim 54, wherein said lectin affinity hemodialysis treatment is administered prior to administering said course of antiviral therapy.

56. The method of claim 54, wherein said lectin affinity hemodialysis treatment is administered concurrent with the administering said course of antiviral therapy.

57. The method of claim 54, wherein said enhancing the efficacy of said antiviral therapy comprises increasing a viral load reduction rate of said individual during said administration of said course of antiviral therapy as compared to a viral load reduction rate achieved by administering either of said lectin affinity hemodialysis treatment or said course of antiviral therapy alone.

58. The method of claim 57, wherein said increase in the viral load reduction rate of said individual is at least about 15%.

59. The method of claim 57, wherein said viral load reduction rate of said individual is at least 15% higher as compared to the viral load reduction rate calculated by combining the viral load reduction rate achieved by administering said lectin affinity hemodialysis treatment alone and the viral load reduction rate achieved by administering said course of antiviral therapy alone.

60. The method of claim 57, wherein said viral load reduction rate of said individual is at least about 20% per day.

61. The method of claim 54, wherein said enhancing the efficacy of said antiviral therapy comprises reducing the amount of time required to achieve a clinically relevant viral load in said individual during said administration of said course of said antiviral therapy as compared to the amount of time required to achieve said clinically relevant viral load by administering either of said lectin affinity hemodialysis treatment or said course of antiviral therapy alone.

62. The method of claim 61, wherein said clinically relevant viral load is less than about 50000 copies/ml.

63. The method of claim 61, wherein said amount of time required to achieve said clinically relevant viral load is reduced by at least about 15%.

64. The method of claim 61, wherein said amount of time required to achieve said clinically relevant viral load is less than about 20 days.

65. The method of claim 54, wherein said course of antiviral therapy comprise administering to said individual at least one antiviral agent, wherein said antiviral agent is selected from the group consisting of immunostimulators, immunomodulators, nucleoside antiviral agents, nucleotide antiviral agents, protease inhibitors, inosine 5′-monophosphate dehydrogenase (IMPDH) inhibitors, viral entry inhibitors, viral maturation inhibitors, viral uncoating inhibitors, integrase inhibitors, viral enzyme inhibitors, anti-sense molecules, ribozyme antiviral agents, nanoviricides, and antibodies.

66. The method of claim 54, wherein said viral infection is caused by a virus selected from the group consisting of ebola virus, marburg virus, smallpox virus, lassa virus, dengue virus, rift valley virus, west nile virus, influenza A virus, H5N1 influenza virus, H1N1 influenza virus, measles virus, mumps virus, viral encephalitis, monkeypox virus, camelpox virus, vaccinia virus, HIV, HCV, hepatitis virus, human cytomegalovirus (HCMV) and distemper virus.

67. The method of claim 66, wherein said viral infection is HIV infection.

68. The method of claim 66, wherein said viral infection is HCV infection.

69. The method of claim 54, wherein the lectin affinity hemodialysis treatment is administered for a period selected from the group consisting of about 1 to 8 hours during a 24 hour period.

70. The method of claim 54, wherein said hemodialysis treatment is administered no more than four times per week.

71. A kit for treating an individual suffering from viral infection, the kit comprising:
a lectin affinity hemodialysis device; and
at least one antiviral agent.

72. An enhanced lectin affinity hemodialysis therapy method for treating an individual suffering from viral infection, the method comprising:
administering a course of lectin affinity hemodialysis therapy to said individual, wherein said lectin affinity hemodialysis therapy comprises passing blood or plasma from said individual through a lectin affinity hemodialysis device, wherein a lectin in said lectin affinity hemodialysis device binds a virus or fragments thereof in said blood or plasma, and wherein said lectin traps said virus or fragments thereof in said lectin affinity hemodialysis device, removing said virus or fragments thereof from said blood or plasma; and
enhancing the efficacy of said course of lectin affinity hemodialysis therapy by administering to said individual a course of antiviral therapy during the course of said hemodialysis treatment.

73. The method of claim 72, wherein said enhancement of said course of lectin affinity hemodialysis therapy comprises reducing the average viral load during the course of said lectin affinity hemodialysis therapy.
